(19) United States
(12) Patent Application Publication
(10) Pub. No.: US 2011/0015130 A1
(43) Pub. Date: Jan. 20, 2011

(54) Polypeptides Selective for alphavbeta3 Integrin Conjugated With a Variant Of Human Serum Albumin (HSA) And Pharmaceutical Uses Thereof

(76) Inventors: Woei-Jer Chuang, Tainan (TW); Wen-Mei Fu, Taipei (TW)

Correspondence Address: Wood, Phillips, Katz, Clark & Mortimer;Citigroup Center, Suite 3800, 500 West Madison Street, Chicago, IL, 60661, US

(21) Appl. No.: 12/838,926

(22) Filed: Jul. 19, 2010

Related U.S. Application Data
(60) Provisional application No. 61/226,945, filed on Jul. 20, 2009.

(51) Int. Cl. A61K 38/38; C07K 14/765; C12P 21/02; A61P 27/02; A61P 19/10; A61P 3/14; A61P 35/00; A61P 35/04; A61P 29/00

(52) U.S. Cl. 514/15.2; 530/363; 435/69.6

(57) ABSTRACT

The invention generally relates to fusion proteins comprising a rhodostomin variant having an RGD motif variant ⁴⁸ARLDDL⁵³, wherein the rhodostomin variant is conjugated with a variant of Human Serum Albumin (HSA). The invention also relates to the use of these fusion proteins for treatment and prevention of αvβ3 integrin-associated diseases.

### FIELD OF THE INVENTION

The present invention generally relates to fusion proteins comprising a rhodostomin variant having an RGD motif variant ⁴⁸ARLDDL⁵³, wherein the rhodostomin variant is conjugated with a variant of Human Serum Albumin (HSA). The invention also relates to the use of these fusion proteins for the treatment and prevention of αvβ3 integrin-associated diseases.

### BACKGROUND OF THE INVENTION

Bone is a complex tissue composed of several cell types which are continuously undergoing a process of renewal and repair termed “bone remodeling.” The two major cell types responsible for bone remodeling are osteoclasts, which resorb bone, and osteoblasts, which form new bone. Bone remodeling has been known to be regulated by several systemic hormones (e.g., parathyroid hormone, 1,25-dihydroxy vitamin D₃, sex hormones, and calcitonin) and local factors (e.g., nitric oxide, prostaglandins, growth factors, and cytokines).

Integrins are heterodimeric matrix receptors that anchor cells to substrates and transmit externally derived signals across the plasma membrane. Integrin αvβ3 is involved in the osteoclast-mediated bone resorption, both in vivo and in vitro. This heterodimer molecule recognizes the amino acid motif Arg-Gly-Asp (RGD) contained in bone matrix proteins such as osteopontin and bone sialoprotein. Integrin αvβ3 is expressed in an osteoclast and its expression is modulated by resorptive steroids and cytokines. Based on blocking experiments, αvβ3 integrin has been identified as a major functional adhesion receptor on osteoclasts. Inhibitors of integrin αvβ3 reduce the capacity of osteoclasts to bind to and resorb bone. Integrin αvβ3 plays a major role in the function of osteoclasts and inhibitors of this integrin are being considered for treating or preventing osteoporosis, osteolytic metastases, and malignancy-induced hypercalcemia.

There are many bone diseases that are related to osteolysis that is mediated by osteoclasts. Osteoporosis is the most common one that is induced when resorption and formation of bone are not coordinated and bone breakdown overrides bone building. Osteoporosis is also caused by other conditions, such as hormonal imbalance, diseases, or medications (e.g., corticosteroids or anti-epileptic agents). Bone is one of the most common sites of metastasis by human breast, prostate, lung and thyroid cancers, as well as other cancers. Osteoporosis may also result from post-menopausal estrogen deficiency. Secondary osteoporosis may be associated with rheumatoid arthritis. Bone metastasis shows a very unique step of osteoclastic bone resorption that is not seen in metastasis of other organs. It is widely accepted that osteolysis that is associated with cancer is essentially mediated by osteoclasts, which seem to be activated and may be indirectly activated through osteoblasts or directly by tumor products. In addition, hypercalcemia (increased blood-calcium concentration) is an important complication of osteolytic bone diseases. It occurs relatively frequently in patients with extensive bone destruction, and is particularly common in breast, lung, renal, ovarian and pancreatic carcinomas and in myeloma.

Disintegrins are a family of low-molecular-weight RGD-containing peptides that bind specifically to integrins αllbβ3, α5β1 and αvβ3 expressed on platelets and other cells including vascular endothelial cells and some tumor cells. In addition to their potent antiplatelet activity, studies of disintegrins have revealed new uses in the diagnosis of cardiovascular diseases and the design of therapeutic agents in arterial thrombosis, osteoporosis and angiogenesis-related tumor growth and metastasis. Rhodostomin (Rho), a disintegrin derived from the venom of Colloselasma rhodostoma, has been found to inhibit platelet aggregation in vivo and in vitro through the blockade of platelet glycoprotein αllbβ3. Furthermore, rhodostomin is reported to inhibit the adhesion of breast and prostate carcinoma cells to both unmineralized and mineralized bone extracellular matrices in a dose-dependent manner, without affecting the viability of tumor cells. In addition, rhodostomin inhibits the migration and invasion of breast and prostate carcinoma cells. Rhodostomin has also been shown to inhibit adipogenesis and obesity. However, because rhodostomin non-specifically binds to integrins αllbβ3, α5β1 and αvβ3, the pharmaceutical uses of rhodostomin may cause serious side effects. For example, when applying rhodostomin in treating carcinomas, the inhibition of platelet aggregation is an undesirable side effect.

The role of αvβ3 integrin in bone diseases has been well documented. See, for example, F. Patrick Ross et al, Nothing but skin and bone, the Journal of Clinical Investigation, Vol. 116, #5, May 2006; S. B. Rodan et al, Integrin function in osteoclasts, Journal of Endocrinology (1997) 154, S47-S56; Steven L. Teitelbaum, Editorial: Osteoporosis and Integrins, the Journal of Clinical Endocrinology and Metabolism, April 2005, 90(4): 2466-2468; Steven L. Teitelbaum, Osteoclasts, integrins, and osteoporosis, Journal of Bone and Mineral Metabolism, (2000) 18: 344-349; Ichiro Nakamura et al, Involvement of αvβ3 integrins in osteoclast function, Journal of Bone and Mineral Metabolism, (2007) 25: 337-344; Le T. Duong et al, The role of integrins in osteoclast function, Journal of Bone and Mineral Metabolism, (1999) 17: 1-6; and A Teti et al, The Role of the AlphaVbeta3 Integrin in the Development of Osteolytic Bone Metastases: A Pharmacological Target for Alternative Therapy?, Calcified Tissue International (2002) 71: 293-299.

In addition to bone diseases, αvβ3 integrin plays an important role in angiogenesis and tumor growth in conditions not related to bone diseases.

Thus, it may be desirable to create polypeptides selective for αvβ3 integrin with improved stability and lasting effects. These polypeptides will be potentially suitable to treat diseases and conditions involving αvβ3 integrin, including but not limited to various bone diseases, cancer, and diseases involving angiogenesis.

Human serum albumin (HSA) fusion technology has been used in the art to create long acting protein pharmaceuticals. However, polypeptides conjugated with HSA may be prone to disulfide-linked aggregation, especially in acidic conditions, resulting in the formation of intermolecular dimers. The formation of intermolecular dimers may lower the activity of the polypeptides and/or cause immunogenicity when these polypeptides are administered to mammals.

Accordingly, there is a need in the art to create a polypeptide which is selective for αvβ3 integrin with better stability and fewer intermolecular dimers than the polypeptide fused with wild type HSA.

### SUMMARY OF THE INVENTION

In one embodiment, the invention relates to a polypeptide comprising an amino acid sequence of SEQ ID NO: 1, wherein the polypeptide is conjugated with a variant of human serum albumin (HSA) comprising an amino acid sequence of SEQ ID NO: 4, or a pharmaceutically acceptable salt of said polypeptide.

SEQ ID NO: 1 represents an amino acid sequence of a rhodostomin variant having an RGD motif variant ⁴⁸ARLDDL⁵³.

SEQ ID NO: 2 and SEQ ID NO: 3 represent two of the possible nucleotide sequences that encode a rhodostomin variant having an RGD motif variant ⁴⁸ARLDDL⁵³.

SEQ ID NO: 4 represents an amino acid sequence of the HSA variant, wherein the cysteine residue at position 34 of the HSA amino acid sequence has been replaced with serine. This HSA variant is referred to as HSA C34S.

SEQ ID NO: 5 represents a nucleotide sequence that encodes HSA C34S variant.

In another embodiment, the invention relates to a polypeptide comprising an amino acid sequence of SEQ ID NO: 1, wherein the polypeptide is conjugated with a variant of HSA comprising an amino acid sequence of SEQ ID NO: 6, or a pharmaceutically acceptable salt of said polypeptide.

SEQ ID NO: 6 represents an amino acid sequence of the HSA variant, wherein the cysteine residue at position 34 of the HSA amino acid sequence has been replaced with alanine. This HSA variant is referred to as HSA C34A.

SEQ ID NO: 7 represents a nucleotide sequence that encodes HSA C34A variant.

In a preferred embodiment, the invention provides a polypeptide comprising an amino acid sequence of SEQ ID NO: 1, wherein the polypeptide is conjugated with a variant of HSA comprising an amino acid sequence of SEQ ID NO: 4 or SEQ ID NO: 6, wherein said polypeptide further comprises a linker amino acid sequence, or a pharmaceutically acceptable salt of said polypeptide.

In a more preferred embodiment, the linker amino acid sequence comprises a combination of glycine and serine amino acids.

In a more preferred embodiment, the linker amino acid sequence comprises an amino acid sequence of SEQ ID NO: 8.

In the most preferred embodiment, the invention relates to a polypeptide comprising an amino acid sequence of SEQ ID NO: 9, or a pharmaceutically acceptable salt of said polypeptide.

SEQ ID NO: 9 represents an amino acid sequence of HSA(C34S)-ARLDDL fusion protein, wherein ARLDDL rhodostomin variant is fused to HSA C34S variant through the linker amino acid sequence of SEQ ID NO: 8.

In another preferred embodiment, the invention relates to a polypeptide comprising an amino acid sequence of SEQ ID NO: 11.

SEQ ID NO: 11 represents an amino acid sequence of HSA(C34A)-ARLDDL fusion protein, wherein ARLDDL rhodostomin variant is fused to HSA C34A variant through the linker amino acid sequence of SEQ ID NO: 8.

In one embodiment, the invention relates to a polypeptide encoded by a polynucleotide comprising a nucleotide sequence of SEQ ID NO: 10.

In another embodiment, the invention relates to a polypeptide encoded by a polynucleotide comprising a nucleotide sequence of SEQ ID NO: 12.

Because of the degeneracy of the genetic code, it is within the skill in the art to modify the nucleotide sequences of SEQ ID NO: 10 and SEQ ID NO: 12 to create other polynucleotides encoding the polypeptides of the present invention. Therefore, the polypeptides of the present invention encoded by other polynucleotides are also encompassed by the present invention.

The polypeptides of the present invention are generally highly selective for αvβ3 integrin and exhibit reduced binding to αllbβ3 and/or α5β1 integrin as compared to a wild type disintegrin.

The polypeptides of the present invention generally exhibit at least about a 5, 50, or 100-fold decrease in affinity to αllbβ3 and/or α5β1 as compared to rhodostomin.

In another embodiment, the polypeptides of the present invention generally exhibit at least about a 200-fold decrease in affinity to αllbβ3 integrin as compared to rhodostomin, more preferably at least about 500-fold decrease in affinity to αllbβ3 integrin as compared to rhodostomin,

In another embodiment, the polypeptides of the present invention generally exhibit at least about a 20-fold decrease in affinity to α5β1 integrin as compared to rhodostomin, and more preferably, at least about a 70 or 90-fold decrease in affinity to α5β1 integrin as compared to rhodostomin.

The polypeptides of the present invention generally exhibit at least about 5, 50, 100, or 150-fold decrease in affinity to platelets as compared to rhodostomin.

In still another embodiment of the invention, the polypeptides exhibit a substantially reduced activity in prolongation of blood clotting time as compared to rhodostomin and/or a wild type disintegrin.

In another embodiment, the invention relates to a physiologically acceptable composition comprising a polypeptide of the invention, or a pharmaceutically acceptable salt thereof, and a pharmaceutically acceptable carrier.

In a preferred embodiment, the invention relates to a physiologically acceptable composition comprising a polypeptide comprising an amino acid sequence of SEQ ID NO: 9, or a pharmaceutically acceptable salt of said polypeptide, and a pharmaceutically acceptable carrier.

In another preferred embodiment, the invention relates to a physiologically acceptable composition comprising a polypeptide encoded by a polynucleotide comprising a nucleotide sequence of SEQ ID NO: 10, or a pharmaceutically acceptable salt of said polypeptide, and a pharmaceutically acceptable carrier.

In another embodiment, the invention relates to a method for treatment and/or prevention of an αvβ3 integrin-associated disease comprising administering to a mammal in need thereof a therapeutically effective amount of a polypeptide comprising an amino acid sequence of SEQ ID NO: 1, wherein the polypeptide is conjugated with a variant of HSA comprising an amino acid sequence of SEQ ID NO: 4 or SEQ ID NO: 6, or a pharmaceutically acceptable salt of said polypeptide.

In a preferred embodiment, the invention relates to a method for the treatment and/or prevention of an αvβ3 integrin-associated disease comprising administering to a mammal in need thereof a therapeutically effective amount of a polypeptide comprising an amino acid sequence of SEQ ID NO: 9, or a pharmaceutically acceptable salt of said polypeptide.

In another preferred embodiment, the invention relates to a method for the treatment and/or prevention of an αvβ3 integrin-associated disease comprising administering to a mammal in need thereof a therapeutically effective amount of a polypeptide encoded by a polynucleotide comprising a nucleotide sequence of SEQ ID NO: 10, or a pharmaceutically acceptable salt of said polypeptide.

In one embodiment of the invention, the αvβ3 integrin-associated disease includes, but is not limited to, osteoporosis, bone tumor or cancer growth and symptoms related thereto, angiogenesis-related tumor growth and metastasis, tumor metastasis in bone, malignancy-induced hypercalcemia, Paget's disease, ovariectomy-induced physiological change, rheumatic arthritis, osteoarthritis and angiogenesis-related eye disease, including but not limited to, age-related macular degeneration, diabetic retinopathy, corneal neovascularizing diseases, ischaemia-induced neovascularizing retinopathy, high myopia and retinopathy of prematurity.

In another embodiment, the invention relates to a method of using a polypeptide of the invention for the inhibition and/or prevention of tumor cell growth in bone or other organs and symptoms related thereto in a mammal.

In another embodiment, the method of treatment and/or prevention of an αvβ3 integrin-associated disease comprises administering to a mammal in need thereof a therapeutically effective amount of a polypeptide comprising an amino acid sequence of SEQ ID NO: 9, or a pharmaceutically acceptable salt of said polypeptide in combination with a therapeutically effective amount of another active agent. The other active agent may be administered before, during or after administering the polypeptide of the present invention.

In a preferred embodiment, the other active agent is selected from the group consisting of VEGF antagonists, anti-inflammation agents, bisphosphonates and cytotoxic agents.

In another embodiment, the invention relates to a method for making a polypeptide of the invention, comprising (a) constructing a gene encoding the polypeptide of the invention; (b) transfecting a host cell with the gene of step (a); (c) growing said host cell in a culture medium; and (d) isolating said polypeptide.

In a preferred embodiment, the invention relates to a method for making a polypeptide comprising an amino acid sequence of SEQ ID NO: 9, comprising (a) constructing a gene encoding said polypeptide; (b) transfecting a host cell with the gene of step (a); (c) growing said host cell in a culture medium; and (d) isolating said polypeptide.

The methods of making the polypeptides of the present invention may further comprise growing a host cell in a culture medium free of amino acids; and collecting the supernatant to obtain said polypeptide.

These methods may further comprise adding methanol to the culture medium to induce polypeptide expression in the host cells.

The methods may further comprise the step of performing column chromatography to obtain said polypeptide.

In one embodiment, the methods may further comprise the step of performing High Performance Liquid Chromatography (HPLC) to obtain the isolated polypeptide.

These and other aspects will become apparent from the following description of the various embodiments taken in conjunction with the following drawings, although variations and modifications therein may be affected without departing from the spirit and scope of the novel concepts of the disclosure.

It is to be understood that both the foregoing general description and the following detailed description are exemplary and explanatory only and are not restrictive of the invention, as claimed.

### BRIEF DESCRIPTION OF THE DRAWINGS

FIGS. 1A and 1B show HPLC profiles of HSA-ARLDDL and HSA(C34S)-ARLDDL, respectively.

FIGS. 1C and 1D show size exclusion chromatography (SEC) profiles of HSA-ARLDDL and HSA(C34S)-ARLDDL, respectively.

FIGS. 1E and 1F show photographs of SDS-PAGE profiles of HSA-ARLDDL and HSA(C34S)-ARLDDL, respectively.

FIG. 1G shows a photograph of 2D SDS-PAGE profiles of HSA-ARLDDL, HSA(C34S)-ARLDDL and HSA.

FIG. 1H shows an NMR spectra of HSA(C34S)-ARLDDL and BSA.

FIG. 2 shows an amino acid sequence SEQ ID NO: 1 of ARLDDL variant of rhodostomin.

FIG. 3A shows a nucleotide sequence SEQ ID NO: 2 of ARLDDL variant of rhodostomin.

FIG. 3B shows a nucleotide sequence SEQ ID NO: 3 of ARLDDL variant of rhodostomin.

FIGS. 4A and 4B show an amino acid sequence SEQ ID NO: 4 and a nucleotide sequence SEQ ID NO: 5 of HSA C34S mutant, respectively.

FIGS. 5A and 5B show amino acid sequence SEQ ID NO: 6 and a nucleotide sequence SEQ ID NO: 7 of HSA C34A mutant, respectively.

FIG. 6 shows an amino acid sequence SEQ ID NO: 8 of a linker amino acid.

FIGS. 7A and 7B show an amino acid sequence SEQ ID NO: 9 and a nucleotide sequence SEQ ID NO: 10 of HSA(C34S)-ARLDDL, respectively.

FIGS. 8A and 8B show an amino acid sequence SEQ ID NO: 11 and a nucleotide sequence SEQ ID NO: 12 of HSA(C34A)-ARLDDL, respectively.

FIGS. 9A, 9B and 9C are photographs of hematopoetic cells of bone marrow showing that HSA(C34S)-ARLDDL inhibits the differentiation of osteoclasts.

FIGS. 10A, 10B and 10C are graphs showing that HSA-ARLDDL and HSA(C34S)-ARLDDL inhibit the differentiation of osteoclasts.

FIGS. 11A, 11B, 11C and 11D are graphs showing that HSA-ARLDDL and HSA(C34S)-ARLDDL inhibit angiogenesis in a mouse model of retinopathy of prematurity (ROP).

FIGS. 11E, 11F and 11G are photographs showing angiogenesis in a mouse model of oxygen-induced retinopathy. They show that HSA(C34S)-ARLDDL inhibits angiogenesis in an oxygen-induced retinopathy mouse.

FIGS. 12A and 12B are photographs of mice injected with the human PC-3 tumor cells. FIG. 12A is control and FIG. 12B shows two mice treated with HSA(C34S)-ARLDDL.

FIGS. 12C and 12D are photographs of tumors excised, respectively, from control mice and mice treated with HSA(C34S)-ARLDDL.

FIG. 13 is a graph that show that HSA(C34S)-ARLDDL significantly reduced tumor size and tumor weight in mice injected with the human PC-3 tumor cells.

FIG. 14A is a set of photographs showing a reduced blood vessel density in MATRIGEL™ plugs from C57BL/6 mice treated with HSA(C34S)-ARLDDL in comparison with untreated control mice.

FIG. 14B is a graph showing a reduced hemoglobin content in MATRIGEL™ plugs from C57BL/6 mice treated with HSA(C34S)-ARLDDL in comparison with untreated control mice.

### DETAILED DESCRIPTION OF THE INVENTION

Various embodiments of the invention are now described in detail. As used in the description and throughout the claims, the meaning of “a”, “an”, and “the” includes plural reference unless the context clearly dictates otherwise. Also, as used in the description and throughout the claims, the meaning of “in” includes “in” and “on” unless the context clearly dictates otherwise. Additionally, some terms used in this specification are more specifically defined below.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. The invention is not limited to the various embodiments given in this specification.

Unless otherwise defined, all technical and scientific terms used herein have the same meaning as commonly understood by one of ordinary skill in the art to which this invention pertains. In the case of conflict, the present document, including definitions will control.

“Around,” “about” or “approximately” shall generally mean within 20 percent, within 10 percent, within 5, 4, 3, 2 or 1 percent of a given value or range. Numerical quantities given are approximate, meaning that the term “around,” “about” or “approximately” can be inferred if not expressly stated.

The terms “polynucleotide,” “nucleotide,” “nucleic acid,” “nucleic acid molecule,” “nucleic acid sequence,” “polynucleotide sequence” and “nucleotide sequence” are used interchangeably to refer to polymeric forms of nucleotides of any length. The polynucleotides can comprise deoxyribonucleotides, ribonucleotides, and/or their analogs or derivatives. The term includes variants. Variants may include insertions, additions, deletions or substitutions. Nucleotide sequences are listed in the 5′ to 3′ direction.

The terms “polypeptide,” “peptide,” and “protein,” used interchangeably to refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic or depsibicyclic peptide backbones. The term includes single chain protein as well as multimers.

The terms also include fusion proteins, including, but not limited to, glutathione S-transferase (GST) fusion proteins, fusion proteins with a heterologous amino acid sequence such as bioluminescent proteins, for example, luciferin, or aequorin (green fluorescent protein), with heterologous and homologous leader sequences, fusion proteins with or without N-terminal methionine residues, pegylated proteins, and immunologically tagged, or his-tagged proteins. Such fusion proteins also include fusions to epitopes. Such fusion proteins can comprise multimers of the peptides of the invention, e.g. homodimers or homomultimers, and heterodimers and heteromultimers. The term also includes peptide aptamers.

The term “hybridizes specifically,” in the context of a polynucleotide, refers to hybridization under stringent conditions. Conditions that increase stringency of both DNA/DNA and DNA/RNA hybridization reactions are widely known and published in the art. Examples of stringent hybridization conditions include hybridization in 4× sodium chloride/sodium citrate (SSC), at about 65-70° C., or hybridization in 4×SSC plus 50% formamide at about 42-50° C., followed by one or more washes in 1×SSC, at about 65-70° C.

The term “ligand” refers to a molecule that binds to another molecule, including a receptor.

The term “mammal” includes, but is not limited to, a human.

The term “host cell” is an individual cell or cell culture which can be or has been a recipient of any recombinant vector(s) or polynucleotide. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention may be called a “recombinant host cell.”

The term “treatment” refers to any administration or application of remedies for disease in a mammal and includes inhibiting the disease, arresting its development, relieving the disease, for example, by causing regression, or restoring or repairing a lost, missing, or defective function; or stimulating an inefficient process. The term includes obtaining a desired pharmacologic and/or physiologic effect, covering any treatment of a pathological condition or disorder in a mammal. The effect may be prophylactic in terms of completely or partially preventing a disorder or symptom thereof and/or may be therapeutic in terms of a partial or complete cure for a disorder and/or adverse affect attributable to the disorder. It includes (1) preventing the disorder from occurring or recurring in a subject who may be predisposed to the disorder but is not yet symptomatic, (2) inhibiting the disorder, such as arresting its development, (3) stopping or terminating the disorder or at least its associated symptoms, so that the host no longer suffers from the disorder or its symptoms, such as causing regression of the disorder or its symptoms, for example, by restoring or repairing a lost, missing or defective function, or stimulating an inefficient process, or (4) relieving, alleviating or ameliorating the disorder, or symptoms associated therewith, where ameliorating is used in a broad sense to refer to at least a reduction in the magnitude of a parameter, such as inflammation, pain and/or tumor size.

The term “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

The term “composition” refers to a mixture that usually contains a carrier, such as a pharmaceutically acceptable carrier or excipient that is conventional in the art and which is suitable for administration into a subject for therapeutic, diagnostic, or prophylactic purposes. It may include a cell culture in which the polypeptide or polynucleotide is present in the cells or in the culture medium. For example, compositions for oral administration can form solutions, suspensions, tablets, pills, capsules, sustained release formulations, oral rinses or powders.

The term “disease” refers to any condition, infection, disorder or syndrome that requires medical intervention or for which medical intervention is desirable. Such medical intervention can include treatment, diagnosis and/or prevention.

The abbreviation “Rho” means “rhodostomin,” which is a disintegrin derived from the venom of Colloselasma rhodostoma. Rhodostomin non-specifically binds to integrins αllbβ3, α5β1 and αvβ3, and prolongs blood clotting time by inhibiting platelet aggregation through the blockade of platelet glycoprotein αllbβ3.

The term “IC₅₀,” or “the half maximal inhibitory concentration” refers to the concentration of Rho or its variant that is required for 50% inhibition of its receptor. IC₅₀ is a measure of how much of Rho or its variant is needed to inhibit a biological process by 50%, such as the variant's affinity to its receptor.

The term “therapeutically effective amount” refers to an amount which, when administered to a living subject, achieves a desired effect on the living subject. For example, an effective amount of the polypeptide of the invention for administration to the living subject is an amount that prevents and/or treats an integrin αvβ3-mediated disease. The exact amount will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art.

The term “receptor antagonist” refers to a binding ligand of a receptor that inhibits the function of a receptor by blocking the binding of an agonist to the receptor, or which allows agonist binding, but inhibits the ability of the agonist to activate the receptor.

The term “substantially reduced integrin αllbβ3 and/or α5β1 receptor-blocking activity” refers to a reduced activity of at least five fold in blocking integrin αllbβ3 and/or α5β1 receptor as compared to wild type rhodostomin or other disintegrins. For example, to calculate the reduction in αllbβ3 and/or α5β1 receptor-blocking activity, the IC₅₀ of a rhodostomin variant for inhibition of integrin αllbβ3 and/or α5β1 binding to a matrix protein, such as fibrinogen, is compared to of the IC₅₀ of Rho.

The term “RGD motif variant” refers to a peptide comprising a modification in the amino acid sequence that spans the RGD sequence of a corresponding wild type sequence, such as the sequence comprising RGD in Rhodostomin.

The term “ARLDDL” refers to a rhodostomin variant having an RGD motif variant ⁴⁸ARLDDL⁵³. The numbers “48” and “53” refer to positions of these amino acids in the amino acid sequence of wild type rhodostomin.

The term “HSA C34S” refers to a human serum albumin (HSA) variant wherein the cysteine residue at position 34 of wild type HSA amino acid sequence has been replaced with serine. HSA C34S comprises SEQ ID NO: 4.

The term “HSA C34A” refers to an HSA variant wherein the cysteine residue at position 34 of wild type HSA amino acid sequence has been replaced with alanine. HSA C34A comprises SEQ ID NO: 6.

The term “HSA(C34S)-ARLDDL” refers to a fusion protein comprising a) a human serum albumin (HSA) variant wherein the cysteine residue at position 34 of wild type HSA amino acid sequence has been replaced with serine, b) the linker amino acid sequence of SEQ ID NO: 8, and c) a rhodostomin variant having an RGD motif variant ⁴⁸ARLDDL⁵³.

HSA(C34S)-ARLDDL is represented by SEQ ID NO: 9.

The term “HSA(C34A)-ARLDDL” refers to a fusion protein comprising a) a human serum albumin (HSA) variant wherein the cysteine residue at position 34 of wild type HSA amino acid sequence has been replaced with alanine, b) the linker amino acid sequence of SEQ ID NO: 8, and a rhodostomin variant having an RGD motif variant ⁴⁸ARLDDL⁵³.

HSA(C34A)-ARLDDL is represented by SEQ ID NO: 11.

The term “inhibitory selectivity for integrin αvβ3 relative to αllbβ3 and/or α5β1 receptors” refers to a polypeptide's binding selectivity toward integrin αvβ3 over αllbβ3 and/or α5β1 receptors, which is expressed as a ratio of the IC₅₀ of the variant for inhibition of αllbβ3 and/or α5β1 receptors over that for inhibition of αvβ3 receptor.

The term “substantially reduced activity in prolongation of blood bleeding time” refers to a polypeptide's reduced ability to inhibit blood clotting in a statistically significant manner as measured by the bleeding time experiment described in the specification.

The terms “pegylated-ARLDDL” or “peg-ARLDDL” refer to a pegylated product of ARLDDL protein.

The terms “albumin-ARLDDL” or “HSA-ARLDDL” refer to a human albumin-conjugated product of ARLDDL protein.

### OVERVIEW OF THE INVENTION

### Selective αvβ3 Disintegrin Variants

U.S. patent application Ser. No. 12/004,045 describes various polypeptides selective for αvβ3 integrin and exhibiting reduced integrin αllbβ3 and/or α5β1 receptor-blocking activity as compared to a wild type disintegrin. These polypeptides are encoded by modified disintegrin nucleotide sequences that encode modified amino acid sequences. As a result, polypeptides are created which have substantially reduced integrin αllbβ3 and/or α5β1 receptor-blocking activity.

Disintegrin variants such as RD-related compounds potently inhibit osteoclast differentiation in vitro. They also inhibit osteoclast resorbing activity and ovariectomy-induced increase in osteoclast formation in animal studies. In addition, RD inhibits the tumor growth of human prostate and breast cancer cells in bone. Malignancy-induced hypercalcemia was also effectively blocked by RD-related proteins. Paget's disease (also known as osteitis deformans) is a chronic bone disorder that typically results in enlarged and deformed bones due to irregular breakdown and formation of bone tissues. Bisphosphonates have been approved for the treatment of Paget's disease. Osteoarthritis is also related to the increase in osteoclast activity. Based on the similar mechanism of action, RD derivatives should also be effective for treatment of these bone disorders. An intravenous injection of RD or PGP at a very large dose at 30 mg/kg did not affect the survival of mice (n=3). In addition, long term administration of PGP (I.V., 0.5 mg/kg/day) for 6 weeks did not affect serum level of creatinine, GOT, and GPT, suggesting lack of side effects on kidney and liver. Therefore, RD and its derivatives, especially ARLDDL, are potential drug candidates for treatment of osteoporosis, bone tumor, malignancy-induced hypercalcemia, Paget's disease, rheumatic arthritis, osteoarthritis and angiogenesis-related eye diseases.

The inventors expressly incorporate by reference all of the disclosure, including the polypeptides, in patent application U.S. Ser. No. 12/004,045.

The present invention is generally related to polypeptides comprising an amino acid sequence of SEQ ID NO: 1, wherein these polypeptides are conjugated with a variant of human serum albumin (HSA), wherein the cysteine residue at position 34 of the HSA amino acid sequence has been replaced either with serine to create HSA C34S mutant protein, or with alanine to create HSA C34A mutant protein.

SEQ ID NO: 1 represents an amino acid sequence of a rhodostomin variant having an RGD motif variant ⁴⁸ARLDDL⁵³.

SEQ ID NO: 2 and SEQ ID NO: 3 represent two of the possible nucleotide sequences that encode a rhodostomin variant having an RGD motif variant ⁴⁸ARLDDL⁵³.

SEQ ID NO: 4 represents an amino acid sequence of the HSA C34S mutant protein.

SEQ ID NO: 5 represents a nucleotide sequence that encodes HSA C34S variant.

SEQ ID NO: 6 represents an amino acid sequence of the HSA C34A mutant protein.

SEQ ID NO: 7 represents a nucleotide sequence that encodes HSA C34A variant.

In a preferred embodiment, the invention provides a polypeptide comprising an amino acid sequence of SEQ ID NO: 1, wherein the polypeptide is conjugated with a variant of HSA comprising an amino acid sequence of SEQ ID NO: 4 or SEQ ID NO: 6, wherein said polypeptide further comprises a linker amino acid sequence, or a pharmaceutically acceptable salt of said polypeptide.

In a preferred embodiment, the linker amino acid sequence comprises a combination of glycine and serine amino acids.

In another preferred embodiment, the linker amino acid sequence comprises an amino acid sequence of SEQ ID NO: 8.

In the most preferred embodiment, the invention relates to a polypeptide comprising an amino acid sequence of SEQ ID NO: 9, or a pharmaceutically acceptable salt of said polypeptide.

SEQ ID NO: 9 represents an amino acid sequence of HSA(C34S)-ARLDDL fusion protein, wherein ARLDDL rhodostomin variant is fused to HSA C34S variant through the linker amino acid sequence of SEQ ID NO: 8.

In another preferred embodiment, the invention relates to a polypeptide comprising an amino acid sequence of SEQ ID NO: 11.

SEQ ID NO: 11 represents an amino acid sequence of HSA(C34A)-ARLDDL fusion protein, wherein ARLDDL rhodostomin variant is fused to HSA C34A variant through the linker amino acid sequence of SEQ ID NO: 8.

In one embodiment, the invention relates to a polypeptide encoded by a polynucleotide comprising a nucleotide sequence of SEQ ID NO: 10.

In another embodiment, a polypeptide of the invention is encoded by a polynucleotide comprising a nucleotide sequence of SEQ ID NO: 12.

Because of the degeneracy of the genetic code, it is within a skill in the art to modify the nucleotide sequences of SEQ ID NO: 10 and SEQ ID NO: 12 to create other polynucleotides encoding the polypeptides of the present invention. Therefore, the polypeptides of the present invention encoded by other polynucleotides are also encompassed by the present invention.

The polypeptides of the invention generally are highly selective for αvβ3 integrin and exhibit reduced binding to αllbβ3 and/or α5β1 integrin compared to a wild type disintegrin.

The polypeptides of the present invention generally exhibit at least about a 5, 50, or 100-fold decrease in affinity to αllbβ3 and/or α5β1 as compared to rhodostomin.

In another embodiment, the polypeptides of the present invention generally exhibit at least about a 200-fold decrease in affinity to αllbβ3 integrin as compared to rhodostomin, more preferably at least about 500-fold decrease in affinity to αllbβ3 integrin as compared to rhodostomin.

The polypeptides of the present invention generally exhibit at least about a 20-fold decrease in affinity to α5β1 integrin as compared to rhodostomin, and more preferably, at least about a 70 or 90-fold decrease in affinity to α5β1 integrin as compared to rhodostomin.

The polypeptides of the present invention generally exhibit at least about 5, 50, 100, or 150-fold decrease in affinity to platelets as compared to rhodostomin.

In still another embodiment of the invention, the polypeptide exhibits a substantially reduced activity in prolongation of blood clotting time as compared to rhodostomin and/or a wild type disintegrin.

### Polypeptides of the Invention

The polypeptides of the invention can be created and expressed using methods known in the art. Cell-based methods and cell-free methods are suitable for producing the polypeptides of the invention. Cell-based methods generally involve introducing a nucleic acid construct into a host cell in vitro and culturing the host cell under conditions suitable for expression, then harvesting the peptide, either from the culture medium or from the host cell, (for example, by disrupting the host cell), or both. The invention also provides methods of producing a peptide using cell-free in vitro transcription/translation methods, which are well known in the art.

Suitable host cells include prokaryotic or eukaryotic cells, including, for example, bacterial, yeast, fungal, plant, insect and mammalian cells.

Example 1 below describes the construction and expression of one polypeptide according to the invention, HSA(C34S)-ARLDDL.

Typically, a polypeptide of the invention may be expressed on its own and may include secretion signals and/or a secretory leader sequence. A secretory leader sequence of the invention may direct certain proteins to the endoplasmic reticulum (ER). The ER separates the membrane-bound proteins from other proteins. Once localized to the ER, proteins can be further directed to the Golgi apparatus for distribution to vesicles, including secretory vesicles, the plasma membrane, lysosomes and other organelles.

Additionally, peptide moieties and/or purification tags may be added to the polypeptides of the invention, in addition to a variant HSA. These peptide moieties and/or purification tags may be removed prior to final preparation of the polypeptide. Suitable purification tags include, for example, V5, polyhistidines, avidin and biotin. Conjugation of peptides to compounds, such as biotin, can be accomplished using techniques well known in the art. (Hermanson ed. (1996) Bioconjugate Techniques; Academic Press). Polypeptides of the invention can also be conjugated with radioisotopes, toxins, enzymes, fluorescent labels, colloidal gold, nucleic acids, vinorelbine and doxorubicin using techniques known in the art. (Hermanson ed. (1996) Bioconjugate Techniques; Academic Press; Stefano et al. (2006).

It may also be possible to create fusion proteins, where the mutant HSA-disintegrin fusion proteins of the present invention are further fused with other proteins. Fusion partners suitable for such use include, for example, fetuin, Fc and/or one or more of their fragments. Polyethylene glycol conjugates with the fusion proteins of the present invention are also provided.

The polypeptides of the invention can also be chemically synthesized using techniques known in the art (e.g., see Hunkapiller et al., Nature, 310:105 111 (1984); Grant ed. (1992) Synthetic Peptides, A Users Guide, W.H. Freeman and Co.; U.S. Pat. No. 6,974,884)). For example, a polypeptide corresponding to a fragment of a polypeptide can be synthesized by use of a peptide synthesizer or through the use of solid-phase methods known in the art.

Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The polypeptides of the invention can be recovered and purified from chemical synthesis and recombinant cell cultures by standard methods which include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In one embodiment, high performance liquid chromatography (“HPLC”) is employed for purification. Well known techniques for refolding protein may be employed to regenerate active conformation when the polypeptide is denatured during isolation and/or purification.

A polypeptide or peptidomimetic of the invention can be further modified with or covalently coupled to one or more of a variety of hydrophilic polymers to increase solubility and circulation half-life of the polypeptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran, and dextran derivatives. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S. (1995) Bioconjugate Chem., 6:150-165; Monfardini, C., et al. (1995) Bioconjugate Chem. 6:62-69; U.S. Pat. Nos. 4,640,835; 4,496,689; 4,301,144; 4,670,417; 4,791,192; 4,179,337, or WO 95/34326.

Polypeptides of the invention can include naturally-occurring and non-naturally occurring amino acids. Polypeptides can comprise D-amino acids, a combination of D- and L-amino acids, and various “designer” or “synthetic” amino acids (for example, β-methyl amino acids, Cα-methyl amino acids, and Nα-methyl amino acids, etc.) to convey special properties. Additionally, polypeptides can be cyclic. Polypeptides can include any known non-classical amino acids. Further, amino acid analogs and peptidomimetics can be incorporated into a polypeptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analogs; amide bond isostere; or tretrazol, and the like.

Further, desamino or descarboxy residues can be incorporated at the terminal ends of the polypeptide to decrease susceptibility to proteases or to restrict conformation.

In one embodiment, C-terminal functional groups of the polypeptides of the present invention include amide, amide lower alkyl, amide di-lower alkyl, lower alkoxy, hydroxy, carboxy, the lower ester derivatives thereof, and the pharmaceutically acceptable salts thereof.

### Pharmaceutical Compositions

In another embodiment, the invention relates to a physiologically acceptable composition comprising a polypeptide of the invention, or a pharmaceutically acceptable salt thereof, and a pharmaceutically acceptable carrier.

In a preferred embodiment, the invention relates to a physiologically acceptable composition comprising a polypeptide comprising an amino acid sequence of SEQ ID NO: 9, or a pharmaceutically acceptable salt of said polypeptide, and a pharmaceutically acceptable carrier.

In another preferred embodiment, the invention relates to a physiologically acceptable composition comprising a polypeptide encoded by a polynucleotide comprising a nucleotide sequence of SEQ ID NO: 10, or a pharmaceutically acceptable salt of said polypeptide, and a pharmaceutically acceptable carrier.

Pharmaceutical compositions of the invention may be provided as formulations with pharmaceutically acceptable carriers, excipients and diluents, which are known in the art. These pharmaceutical carriers, excipients and diluents include those listed in the USP pharmaceutical excipients listing. USP and NF Excipients, Listed by Categories, p. 2404-2406, USP 24 NF 19, United States Pharmacopeial Convention Inc., Rockville, Md. (ISBN 1-889788-03-1). Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as antioxidants, humectants, viscosity stabilizers, and similar agents can be added as necessary. Percutaneous penetration enhancers such as Azone can also be included.

Polypeptides of the invention can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Other formulations for oral or parenteral delivery can also be used, as conventional in the art.

The pharmaceutical compositions of the invention can be formulated into preparations in solid, semi-solid, liquid or gaseous forms, such as tablets, capsules, powders, granules, ointments, solutions, suppositories, injections, inhalants and aerosols.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration.

### Methods of Treatment

In another embodiment, the invention relates to a method for the treatment and/or prevention of an αvβ3 integrin-associated disease comprising administering to a mammal in need thereof a therapeutically effective amount of a polypeptide comprising an amino acid sequence of SEQ ID NO: 1, wherein the polypeptide is conjugated with a variant of HSA comprising an amino acid sequence of SEQ ID NO: 4 or SEQ ID NO: 6, or a pharmaceutically acceptable salt of said polypeptide.

In a preferred embodiment, the invention relates to a method for the treatment and/or prevention of an αvβ3 integrin-associated disease comprising administering to a mammal in need thereof a therapeutically effective amount of a polypeptide comprising an amino acid sequence of SEQ ID NO: 9, or a pharmaceutically acceptable salt of said polypeptide.

In another preferred embodiment, the invention relates to a method for the treatment and/or prevention of an αvβ3 integrin-associated disease comprising administering to a mammal in need thereof a therapeutically effective amount of a polypeptide encoded by a polynucleotide comprising a nucleotide sequence of SEQ ID NO: 10, or a pharmaceutically acceptable salt of said polypeptide.

An αvβ3 integrin-associated disease, includes, but is not limited to, osteoporosis, bone tumor or cancer growth and symptoms related thereto, angiogenesis-related tumor growth and metastasis, tumor metastasis in bone, malignancy-induced hypercalcemia, multiple myeloma, Paget's disease, ovariectomy-induced physiological change, rheumatic arthritis, osteoarthritis and angiogenesis-related eye disease, including but not limited to, age-related macular degeneration, diabetic retinopathy, corneal neovascularizing diseases, ischaemia-induced neovascularizing retinopathy, high myopia and retinopathy of prematurity.

The osteoporosis may be associated with a pathological condition chosen from post-menopausal estrogen deficiency, secondary osteoporosis, rheumatoid arthritis, ovariectomy, Paget's disease, bone cancer, bone tumor, osteoarthritis, increased osteoclast formation and increased osteoclast activity. Furthermore, the osteoporosis includes, but is not limited to, an ovariectomy-induced or post-menopausal osteoporosis, physiological change or bone loss.

In another embodiment, the invention relates to a method of using a polypeptide of the invention for inhibition and/or prevention of tumor cell growth in bone or other organs and symptoms related thereto in a mammal in need thereof.

The pathological symptoms related to tumor cell growth in bone include an increased osteoclast activity, an increased bone resorption, bone lesion, hypercalcemia, a body weight loss, and any combinations thereof. The tumor cell growth in bone includes bone cancer cells and metastasized cancer cells originating from prostate cancer, breast cancer, lung cancer, renal cancer, ovarian cancer, pancreatic cancer or myeloma cancer.

Polypeptides of the invention may be administered to a subject in need of treatment by injection systemically, such as by intravenous injection; or by injection or application to the relevant site, such as by direct injection, or direct application to the site when the site is exposed in surgery; or by topical application.

Polypeptides of the invention can be used as monotherapy. Alternatively, the polypeptides of the invention can be used in combination with another active agent to treat αvβ3 integrin associated diseases.

In another embodiment, the method of treatment and/or prevention of an αvβ3 integrin-associated disease comprises administering to a mammal in need thereof a therapeutically effective amount of a polypeptide comprising an amino acid sequence of SEQ ID NO: 9, or a pharmaceutically acceptable salt of said polypeptide in combination with a therapeutically effective amount of another active agent. The other active agent may be administered before, during, or after administering the polypeptide of the present invention.

In a preferred embodiment, the other active agent is selected from the group consisting of VEGF antagonists, anti-inflammation agents, bisphosphonates and cytotoxic agents.

Administration of the active agents can be achieved in various ways, including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, intramuscular injection, intravitreous injection, topical application, including but not limited to eye drops, creams, and emulsions, implantation and inhalation.

### Methods of Making Polypeptides

In another embodiment, the invention relates to a method for making a polypeptide of the invention, comprising (a) constructing the gene encoding the polypeptide of the invention; (b) transfecting a host cell with the gene of step (a); (c) growing said host cell in a culture medium; and (d) isolating said polypeptide.

In a preferred embodiment, the invention relates to a method for making a polypeptide comprising an amino acid sequence of SEQ ID NO: 9, comprising (a) constructing a gene encoding said polypeptide; (b) transfecting a host cell with the gene of step (a); (c) growing said host cell in a culture medium; and (d) isolating said polypeptide.

The methods of making the polypeptides of the present invention may further comprise growing host cell in a culture medium free of amino acids; and collecting supernatant to obtain said polypeptide.

These methods may further comprise adding methanol to the culture medium to induce the polypeptide expression in the host cells.

The methods may further comprise the step of performing a column chromatography to obtain said polypeptide.

In one embodiment, the methods may further comprise the step of performing a high performance liquid chromatography (HPLC) to obtain the isolated polypeptide.

The present invention is more particularly described in the following examples that are intended as illustrative only, since many modifications and variations therein will be apparent to those skilled in the art.

Human recombinant RANKL and M-CSF were purchased from R&D Systems (Minneapolis, Minn.). The C-terminal telopeptides of type-I collagen ELISA kit was obtained from Cross Laps (Herlev, Denmark). All other chemicals were obtained from Sigma.

### Example 1

### Construction of a Gene Encoding HSA(C34S)-ARLDDL

### Example 1A

### Construction of a Gene Encoding HSA(C34S)-ARLDDL Via Overlap Extension PCR and Ligation

The structural gene of HSA C34S was constructed using HSA (Invitrogen®, clone ID: IOH23065) as a template. The mutation of C34S was produced by two-step polymerase chain reaction (PCR). The first PCR was amplified with the sense primer containing C34S mutation site and with the antisense primer containing Kpn I, Sac II restriction sites and a TAA stop codon. The second PCR was amplified with the sense primer containing BstB I restriction site and the secretion signal sequence and with the antisense primer containing Kpn I, Sac II restriction sites and a TAA stop codon. The secretion signal sequence of HSA prepro peptide, the α factor prepro peptide from Saccharomyces cerevisiae, or preHSA and pro a factor fusion peptide was used for secretory protein expression. The structural gene of ARLDDL was amplified by PCR with the sense primer containing Kpn I restriction site and the spacer region containing GS sequence and with the antisense primer containing Sac II restriction site and a TAA stop codon. The PCR products of HSA C34S with the secretion signal peptide and Rho ARLDDL mutant with the spacer region were digested using Kpn I restriction enzyme and then were ligated. The resulting gene product was cloned into the BstB I and Sac II sites of the yeast recombination vector. The recombinant plasmid was then transformed into an Escherichia coli XL1-blue strain, and the colonies were selected using the agar plates with low salt LB (1% tryptone, 0.5% yeast extract, 0.5% NaCl, 1.5% agar at pH 7.0) and 25 μg/ml antibiotic Zeocin. The E. coli XL1-blue colonies were picked and the plasmid DNA was isolated and sequenced.

### Example 1B

### Construction of a Gene Encoding HSA(C34S)-ARLDDL Via Gene Synthesis

The DNA encoding secretion signal sequence HSA(C34S)-ARLDDL was synthesized. The secretion signal sequence of HSA prepro peptide, the α factor prepro peptide from Saccharomyces cerevisiae, or preHSA and pro α factor fusion peptide was used for secretory protein expression. The resulting gene product was cloned into the yeast recombination vector with proper restriction site. The recombinant plasmid was then transformed into an Escherichia coli XL1-blue strain, and the colonies were selected using the agar plates with low salt LB (1% tryptone, 0.5% yeast extract, 0.5% NaCl, 1.5% agar at pH 7.0) and 25 μg/ml antibiotic Zeocin. The E. coli XL1-blue colonies were picked and the plasmid DNA was isolated and sequenced.

### Example 2

### Protein Expression and Purification of HSA(C34S)-ARLDDL in P. Pastoris and Characterization of HSA(C34S)-ARLDDL

After the clone was confirmed, a total of 10 μg plasmids were digested with proper restriction enzyme site to linearize the plasmids. Pichia host strain was transformed with the linearized constructs by a heat shock method using a Pichia EasyComp™ kit from Invitrogen®, or electroporation. The transformant integrated at the 5′ AOX1 locus by a single crossover. PCR was used to analyze Pichia integrants to determine whether the HSA(C34S)-ARLDDL gene has been integrated into the Pichia genome. The colonies were selected on agar plates containing YPD (1% yeast extract, 2% peptone, 2% glucose, and 2% agar) and 100 μg/ml Zeocin. A number of clones with multiple copies of HSA(C34S)-ARLDDL gene insertions were selected to pick the clone with the highest protein expression. The resulting recombinant HSA(C34S)-ARLDDL contained 585 amino acids of HSA, a spacer containing 17 amino acid residues, and 68 amino acids of rhodostomin ARLDDL mutant.

The resulting recombinant HSA(C34S)-ARLDDL fusion protein was further purified by HPLC (reverse phase C18 HPLC).

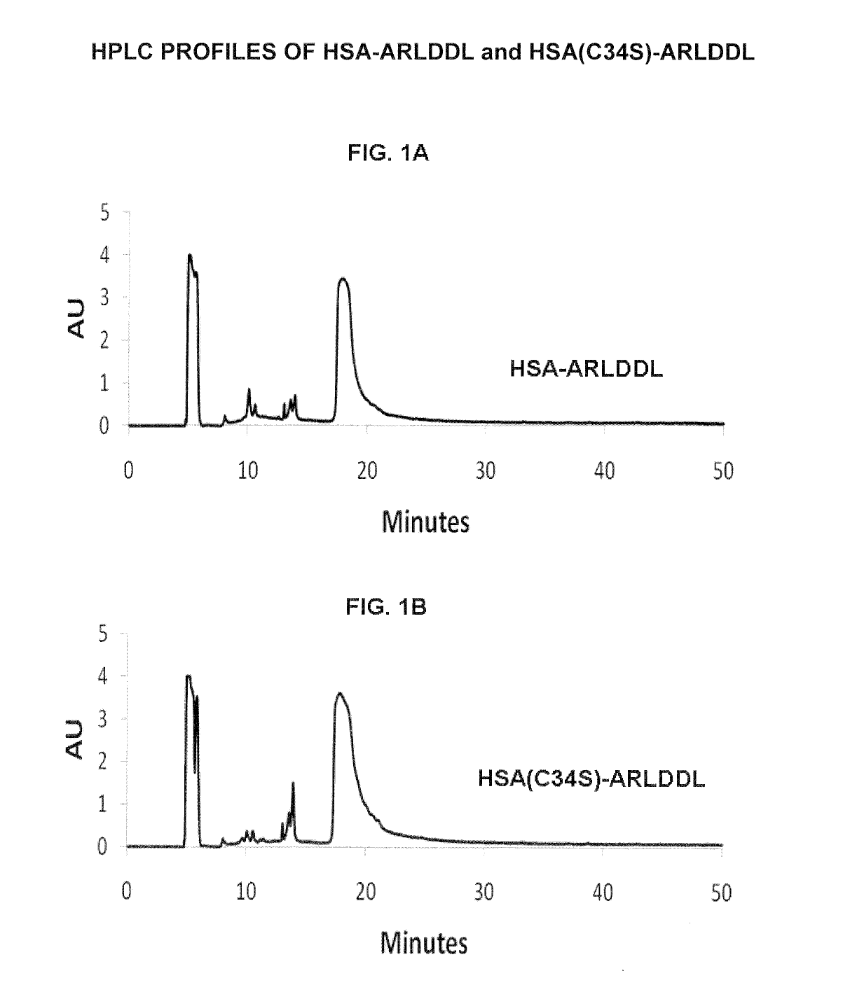

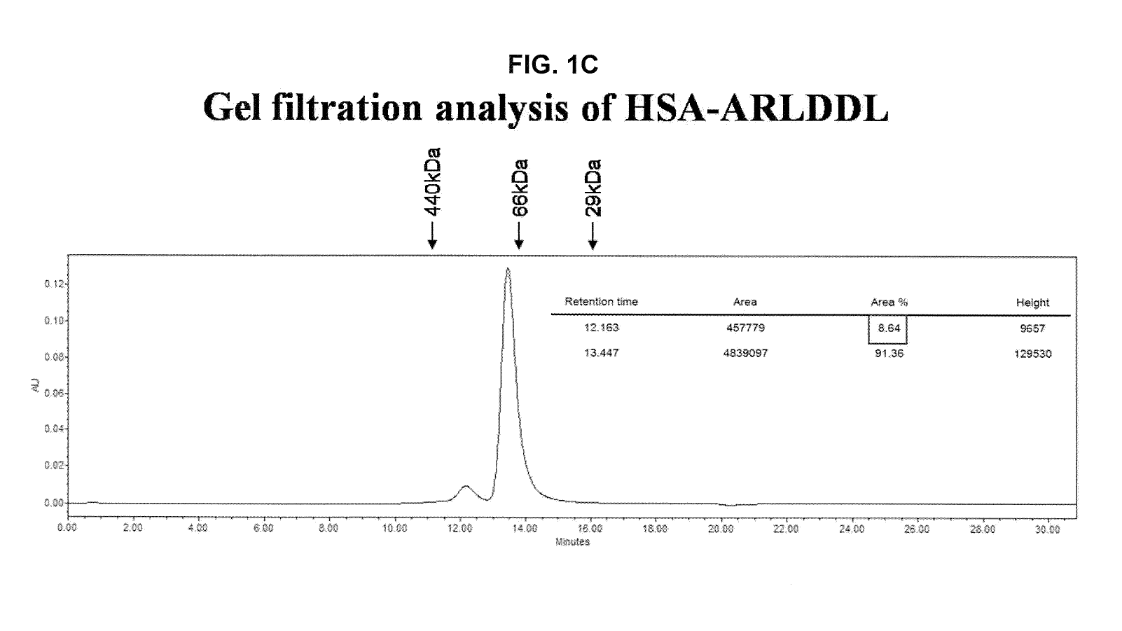

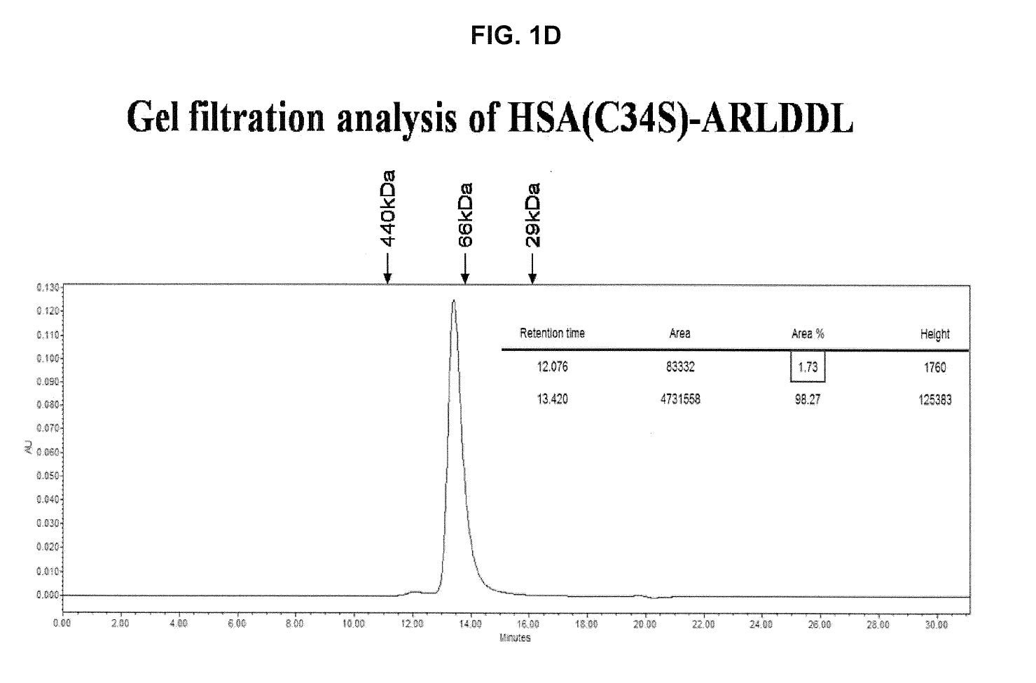

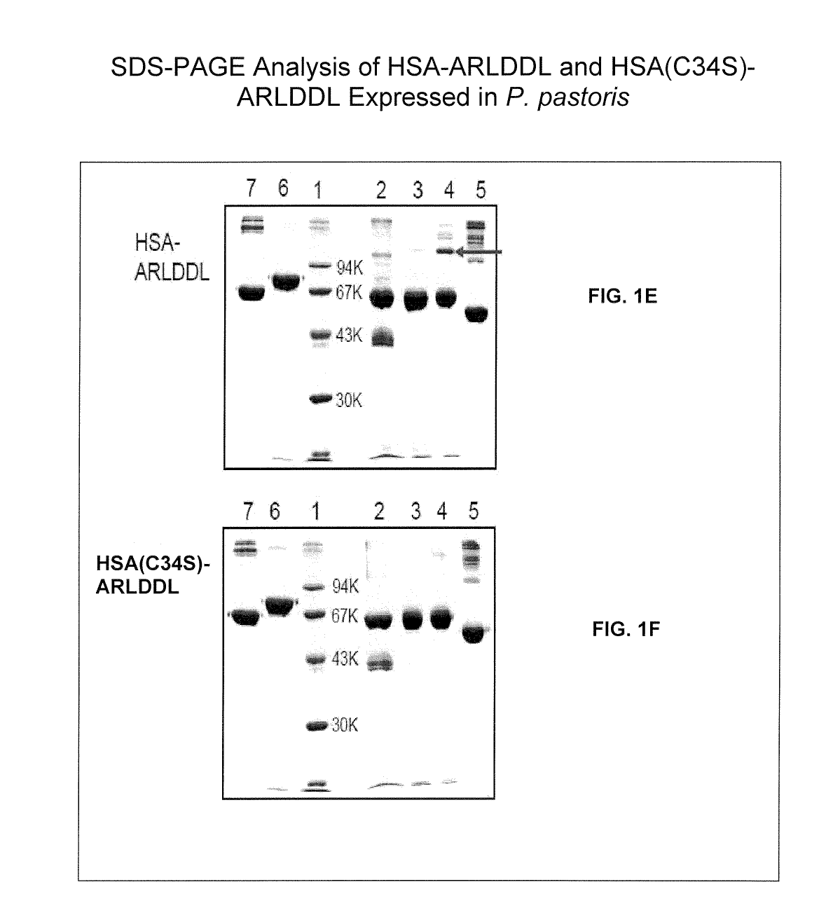

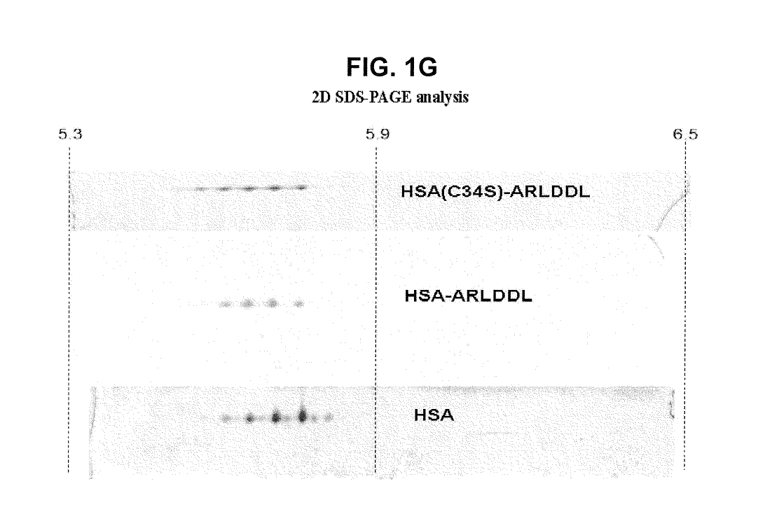

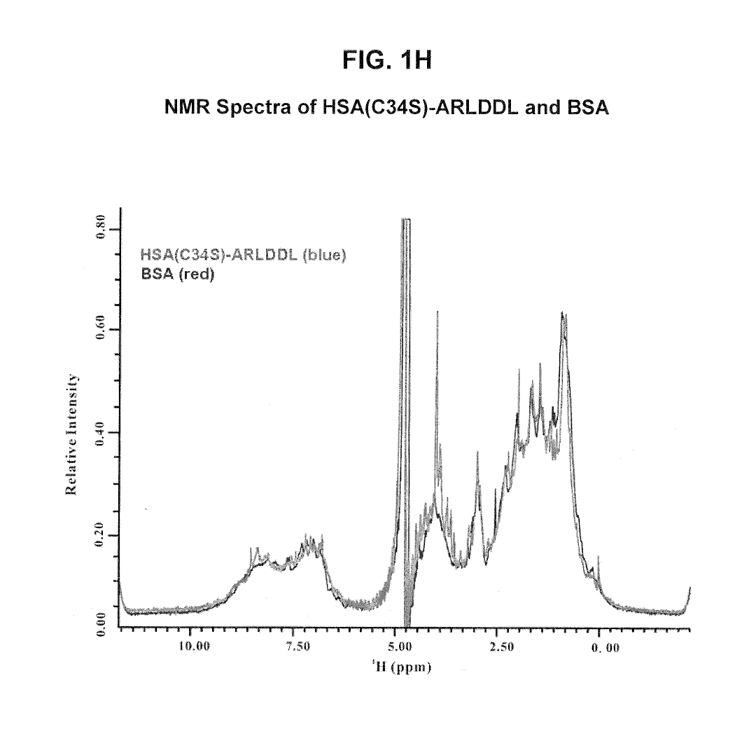

HPLC profiles of HSA-ARLDDL and HSA(C34S)-ARLDDL are shown in FIGS. 1A and 1B, respectively.

The purified recombinant HSA(C34S)-ARLDDL was further analyzed by gel filtration chromatography (also called size exclusion chromatography (SEC)) to separate proteins according to size.

SEC profiles of HSA-ARLDDL and HSA(C34S)-ARLDDL are shown in FIGS. 1C and 1D, respectively. The analysis showed that the mutation at position 34 from C to S in HSA-ARLDDL caused about five-fold decrease in the formation of aggregates.

Table 1 shows the reduction protein aggregates on HSA(C34S)-ARLDDL.

TABLE 1
Reduction of Protein Aggregates in HSA(C34S)-ARLDDL

| | HSA-ARLDDL | HSA(C34S)-ARLDDL |
|---|---|---|
| % of monomers | 91.36 | 98.27 |
| % of aggregates | 8.64 | 1.73 |

FIGS. 1E and 1F show photographs of SDS-PAGE profiles of HSA-ARLDDL and HSA(C34S)-ARLDDL, respectively.

Lane 1 contains Molecular Weight Markers;

Lane 2 contains Methanol Induction;

Lane 3 contains HSA-ARLDDL or HSA(C34S)-ARLDDL purified by blue sepharose chromatography;

Lane 4 contains HSA-ARLDDL or HSA(C34S)-ARLDDL purified by reverse phase HPLC column;

Lane 5 contains commercial BSA;

Lane 6 contains HSA-ARLDDL or HSA(C34S)-ARLDDL purified by blue sepharose chromatography with 2Me; and

Lane 7 contains HSA-ARLDDL or HSA(C34S)-ARLDDL purified by reverse phase HPLC column with 2Me.

The photographs demonstrate that HSA(C34S)-ARLDDL has fewer dimers (represented by red arrow) than HSA-ARLDDL.

HSA(C34S)-ARLDDL, HSA-ARLDDL and human serum albumin (HSA) were also analyzed by 2D SDS-PAGE. 2D SDS-PAGE of HSA(C34S)-ARLDDL, HSA-ARLDDL and HSA are shown in FIG. 1G.

The analysis showed that, similar to HSA, recombinant HSA(C34S)-ARLDDL and HSA-ARLDDL produced from Pichia Pastoris exhibited at least five isoforms in isoelectric focusing dimension.

HSA(C34S)-ARLDDL and bovine serum albumin (BSA) were analyzed by nuclear magnetic resonance (NMR) spectroscopy. FIG. 1H shows the NMR spectra of HSA(C34S)-ARLDDL and BSA. The analysis showed that the folding of HSA(C34S)-ARLDDL was similar to that of BSA. The arrow shows the Hα proton signals from the linker region (G₄S)₅.

### Example 3

### Cell Adhesion Inhibition Assay

Inhibitory Effect on Integrins αvβ3, α5β1, and αllbβ3

The cell adhesion inhibition assay was performed as described in the U.S. patent application Ser. No. 12/004,045. Briefly, wells of 96-well Immulon-2 microtiter plates (Costar, Corning, USA) were coated with 100 μl of phosphate-buffered saline (PBS: 10 mM phosphate buffer, 0.15M NaCl, pH 7.4) containing substrates at a concentration of 50-500 nM, and incubated overnight at 4° C. The substrates and their coating concentrations were fibrinogen (Fg) 200 μg/ml, vitronectin (Vn) 50 μg/ml, and fibronectin (Fn) 25 μg/ml. Non-specific protein binding sites were blocked by incubating each well with 200 μl of heat-denatured 1% bovine serum albumin (BSA, Calbiochem) at room temperature (25° C.) for 1.5 hr The heat-denatured BSA was discarded and each well was washed twice with 200 μl of PBS.

Chinese hamster ovary (CHO) cells expressing αvβ3 (CHO-αvβ3) and αllbβ3 (CHO-αllbβ3) integrins were maintained in 100 μl of Dulbecco's Modified Eagle's Medium (DMEM) medium. Chinese hamster ovary (CHO) cells expressing integrins αvβ3 (CHO-αvβ3) and αllbβ3 (CHO-αllbβ3) were kindly provided by Dr. Y. Takada (Scripps Research Institute). Human erythroleukemia K562 cells were purchased from ATCC and cultured in the RPMI-1640 medium containing 5% fetal calf serum. CHO and K562 cells growing in log phase were detached by trypsinization and used in the assay at 3×105 and 2.5×10⁵ cells/ml, respectively. ARLDDL, pegylated-ARLDDL, HSA-ARLDDL, and HSA(C34S)-ARLDDL were added to the cultured cells and incubated at 37° C., 5% CO2 for 15 minutes. Rho and its variants were used as inhibitors at the concentrations of 0.001-500 μM. The treated cells were then added into the coated plate and reacted at 37° C., 5% CO2 for 1 hour. The incubation solution was then discarded and non-adhered cells were removed by washing twice with 200 μl PBS. Bound cells were quantified by crystal violet staining. Briefly, the well was fixed with 100 μl of 10% formalin for 10 minutes and dried. Fifty microliters of 0.05% crystal violet were then added into the well at room temperature for 20 minutes. Each well was washed with 200 μl of distilled water four times and dried. Colorization was carried out by adding 150 μl of colorizing solution (50% alcohol and 0.1% acetic acid). The resulting absorbance was read at 600 nm and the readings were correlated with the number of adhering cells. Inhibition was defined as % inhibition=100−[OD600 (rhodostomin variant-treated sample)/OD600 (untreated sample)]×100.

### Inhibitory Effect on Platelet Aggregation

The determination of inhibitory effects of ARLDDL and fused proteins on platelet aggregation was performed as described in U.S. patent application Ser. No. 12/004,045.

Briefly, venous blood (9 parts) samples from healthy donors who had not received any medication for at least two weeks were collected in 3.8% sodium citrate (1 part). Blood samples were centrifuged at 150×g for 10 min to obtain platelet-rich plasma (PRP) and allowed to stand for 5 min, and PRP was collected. The platelet-poor plasma (PPP) was prepared from the remaining blood by centrifuging at 2000×g for 25 min. The PPP platelet count was measured on a hematology analyzer and diluted to 250,000 platelets/μl. A solution of 190 μl of PRP and 10 μl of either Rho or PBS buffer were incubated for 5 min in a Henna Tracer 601 aggregometer at 37° C. Ten microliters of 200 μM adenosine diphosphate (ADP) were further added to monitor the response of platelet aggregation by light transmission. The lower the IC₅₀, the greater the specificity or potency of the variant.

### Results

Table 2 demonstrates the inhibitory effects of HSA(C34S)-ARLDDL and other tested proteins on integrins αvβ3, α5β1, and αllbβ3 and on platelet aggregation.

TABLE 2
Inhibition of Platelet Aggregation and Cell Adhesion
by HSA(C34S)-ARLDDL and Other Proteins

| | Platelet | IC₅₀ (nM) | | |
|---|---|---|---|---|
| Proteins | aggregation | αvβ3 | α5β1 | αllbβ3 |
| ARLDDL | 10380 | 48 | 6420 | 23171 |
| Peg-ARLDDL | 13210 | 115 | >5850 | 15672 |
| HSA-ARLDDL | 11234 | 53 | >5850 | 12321 |
| HSA(C34S)- | 10973 | 38 | >5850 | 11432 |
| ARLDDL | | | | |

As Table 2 demonstrates, the C34S modification in the HSA-ARLDDL construct had virtually no effect on the activity in inhibiting the integrin αllbβ3 or α5β1's binding to the matrix proteins. However, the selectivity to the integrin αvβ3 was increased as a result of the sequence modification (IC₅₀ of 38 vs 53).

### Example 4

### Effects of HSA(C34S)-ARLDDL on Osteoclastogenesis

Osteoclasts are specialized monocyte/macrophage family members that differentiate from bone marrow hematopoietic precursors. Cultures of osteoclast precursors in the presence of M-CSF (20 ng/ml) and sRANKL (50 ng/ml) for 8 days induced the formation of large mature osteoclasts with multi-nuclei, which were characterized by the acquisition of mature phenotypic markers, such as TRAP. The method of osteoclastogenesis from cultured hematopoietic cells of bone marrow and the effects of HSA(C34S)-ARLDDL and related proteins on osteoclastogenesis were investigated as follows.

Bone marrow cells were prepared by removing femurs from 6˜8-week-old SD rats and flushing the bone marrow cavity with a-MEM which was supplemented with 20 mM HEPES and 10% heat-inactivated FCS, 2 mM-glutamine, penicillin (100 U/ml) and streptomycin (100 μg/ml). The non-adherent cells (hematopoietic cells) were collected and used as osteoclast precursors after 24 hr. Cells were seeded at 1×106 cells/well (0.5 ml) in 24-well plates in the presence of human recombinant soluble RANKL (50 ng/ml) and murine M-CSF (20 ng/ml). The culture medium was replaced every 3 days. Osteoclast formation was confirmed by an assay of tartrate-resistant acid phosphatase (TRAP) on day-8. In brief, adherent cells were fixed with 10% formaldehyde in phosphate-buffered saline for 3 min. After treatment with ethanol/acetone (50:50 v/v) for 1 min, the cell surface was air-dried and incubated for 10 min at room temperature in an acetate buffer (0.1 M sodium acetate, pH 5.0) containing 0.01% naphthol AS-MX phosphate (Sigma) and 0.03% fast red violet LB salt (Sigma) in the presence of 50 mM sodium tartrate. Osteoclast-like TRAP-positive cells in each well were scored by counting the number of TRAP-positive and multinucleated cells containing more than three nuclei.

HSA(C34S)-ARLDDL and HSA-ARLDDL markedly inhibited the differentiation of osteoclasts.

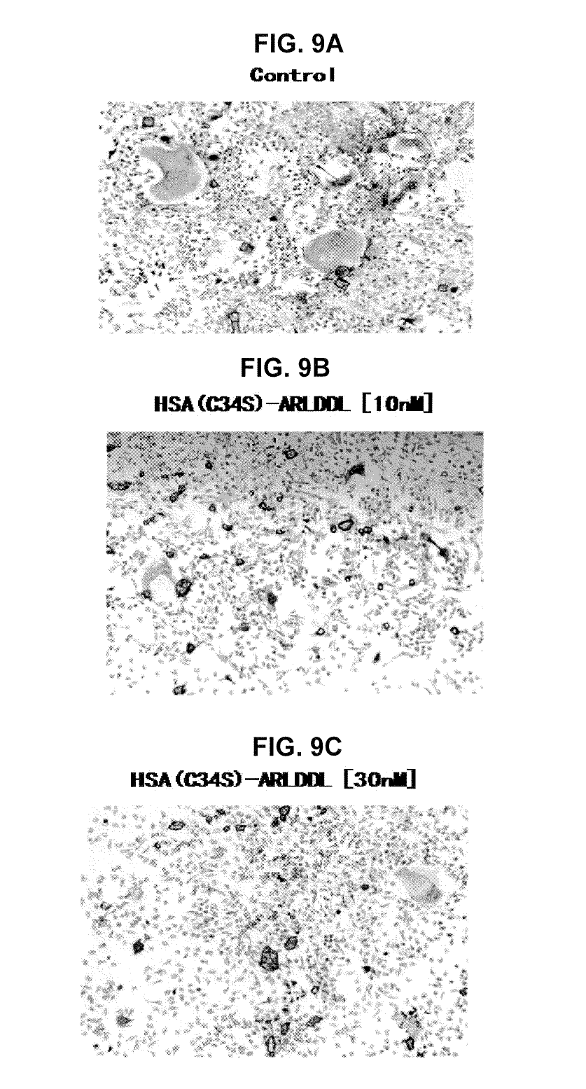

FIG. 9A is control. It demonstrates osteoclasts in cells which were not treated with any polypeptides.

FIG. 9B shows cells treated with 10 nM of HSA(C34S)-ARLDDL.

FIG. 9C shows cells treated with 30 nM of HSA(C34S)-ARLDDL.

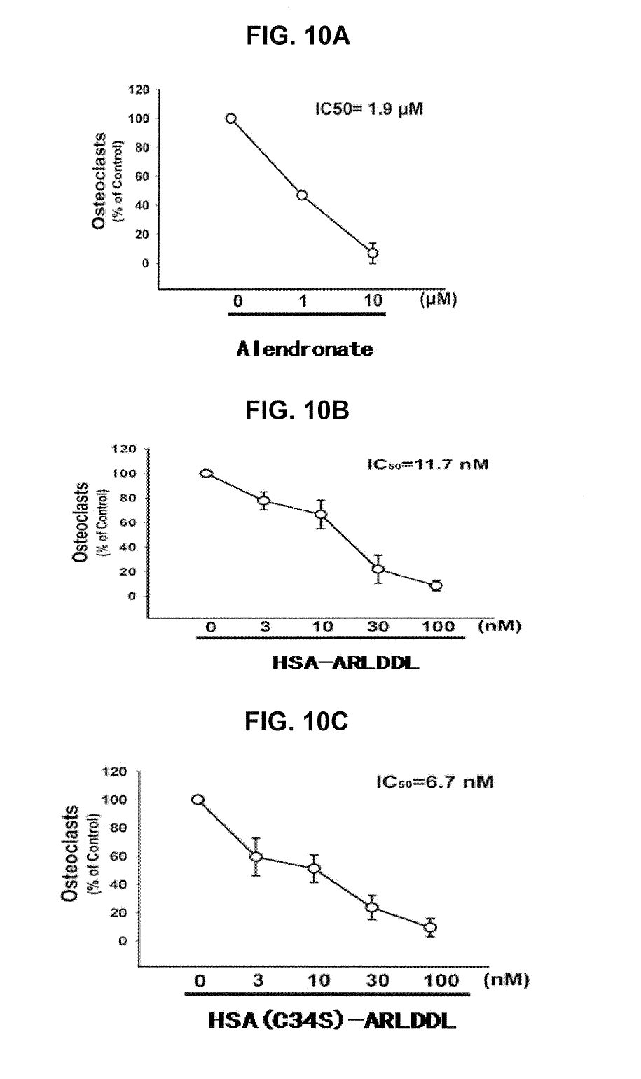

FIG. 10A is a graph demonstrating that as the concentration of alendronate increases, the number of osteoclasts decreases. The IC₅₀ for alendronate was measured to be 1.9 μM.

FIG. 10B is a graph demonstrating that as the concentration of HSA-ARLDDL increases, the number of osteoclasts decreases. The IC₅₀ for HSA-ARLDDL was measured to be 11.7 nM.

FIG. 10C is a graph demonstrating that as the concentration of HSA(C34S)-ARLDDL increases, the number of osteoclasts decreases. The IC₅₀ for HSA(C34S)-ARLDDL was measured to be 6.7 nM.

As this experiment demonstrated, both HSA(C34S)-ARLDDL and HSA-ARLDDL were significantly more effective than alendronate in reducing the number of osteoclasts.

### Example 5

### Inhibition of Angiogenesis by HSA-ARLDDL and HSA(C34S)-ARLDDL in a Mouse Model of Retinopathy of Prematurity

An animal model for retinopathy of prematurity in mice was generated by using hypoxic-induced angiogenesis as described in Wilkinson-Berka et al. (Wilkinson-Berka, J. L., Alousis, N. S., Kelly D. J., et al (2003) COX-2 inhibition and retinal angiogenesis in a mouse model of retinopathy of prematurity. Invest Ophthalmol Vis Sci 44: 974-979.28). Briefly, seven-day-old pups and their mother were housed in sealed chambers containing 75% O₂ and air. Mice remained in the chamber for five days (hyperoxic period, P7 to P12) and were then housed in room air for a further seven days (hypoxic-induced angiogenic period, postnatal 12 days to postnatal 19 days, or P12 to P19). Either HSA-ARLDDL or HSA(C34S)-ARLDDL at various amounts was administered via an intravitreous route on day-12 and the mice were sacrificed on day-19.

Three sections from one of the eyes of each animal were made, deparaffinized, and stained with hematoxylin and eosin. Blood vessel profiles (BVPs) were counted in the inner retina, and included vessels adherent to the inner limiting membrane. Counting was performed on a photomicroscope (Leica) at a magnification of 100×.

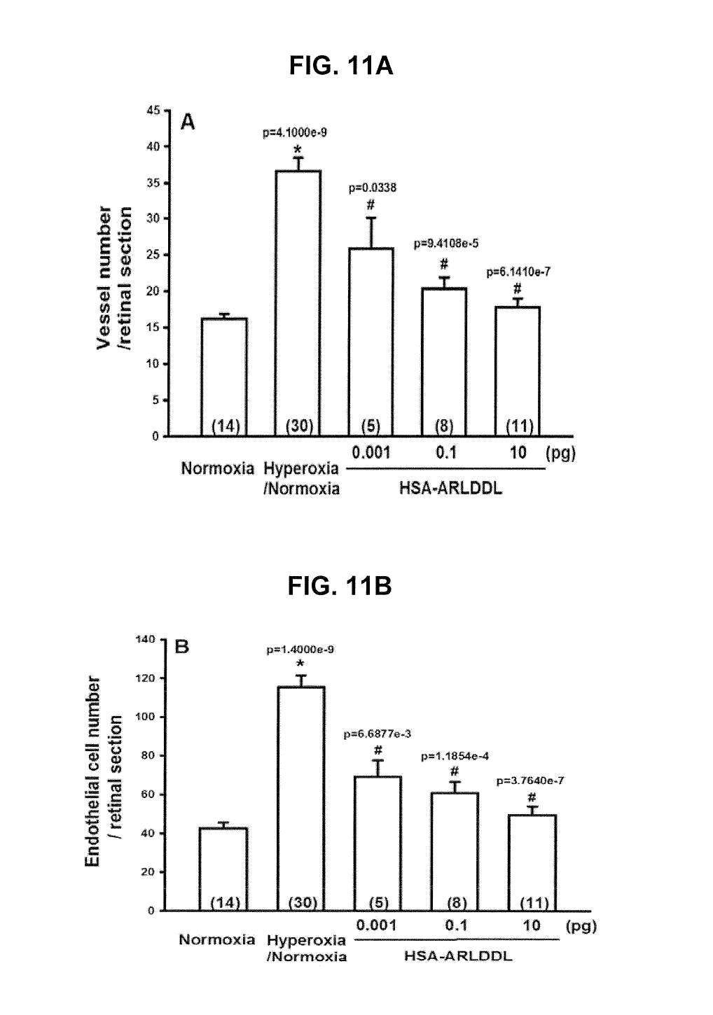

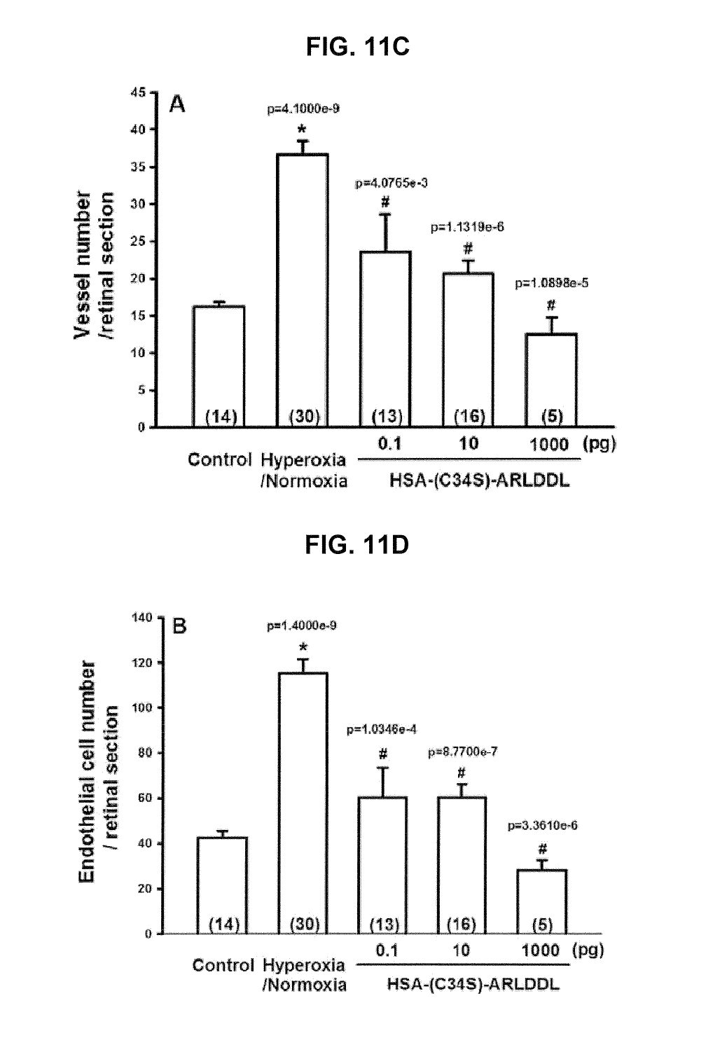

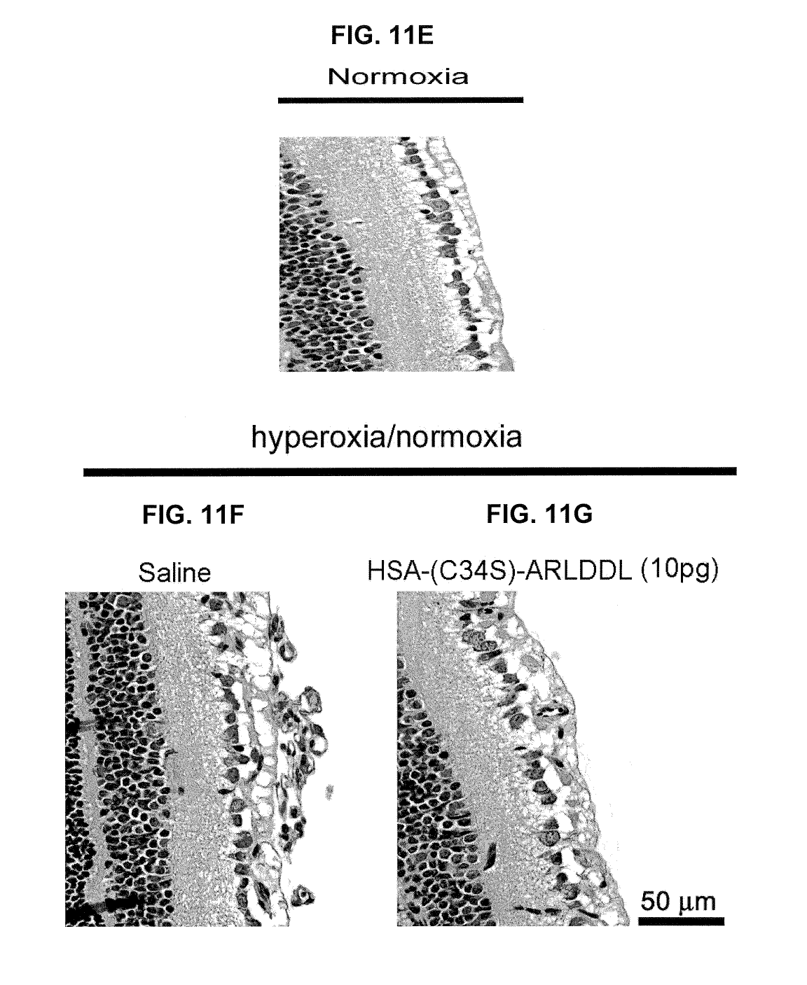

As shown in FIG. 11A, HSA-ARLDDL inhibited angiogenesis in a mouse model of retinopathy of prematurity (ROP). HSA-ARLDDL at doses of 0.001, 0.1 and 10 pg/eye reduced the vessel number per retinal section as compared to normal saline treated group. Data are presented as Mean±SE. With the exception of the group administered 0.001 pg/eye of HSA-ARLDDL, p was less than 0.001.

Endothelial cells were counted in the anterior part of the ganglion cell layer and on inner limiting membrane of the retina by a person blinded to the same identity. The results are shown in FIG. 11B.

The results demonstrate that HSA-ARLDDL at 0.001 pg, 0.1 pg and 10 pg per eye reduced the endothelial cell number per retinal section as compared to the normal saline treated group.

As shown in FIG. 11C, HSA(C34S)-ARLDDL also inhibited angiogenesis in a mouse model of retinopathy of prematurity (ROP). HSA(C34S)-ARLDDL at doses of 0.1, 10 and 1000 pg/eye reduced the vessel number per retinal section as compared to normal saline treated group. Data are presented as Mean±SE. In all instances, p was less than 0.001.

Endothelial cells were counted in the anterior part of the ganglion cell layer and on inner limiting membrane of the retina by a person blinded to the same identity. The results are shown in FIG. 11D.

The results demonstrate that HSA(C34S)-ARLDDL at 0.1 pg, 10 pg and 1000 pg per eye reduced the endothelial cell number per retinal section as compared to the normal saline treated group.

FIGS. 11E, 11F and 11G are photographs showing angiogenesis in a mouse model of oxygen-induced retinopathy. FIG. 11E shows normoxia (control group), FIG. 11F shows angiogenesis in an oxygen-induced retinopathy mouse and FIG. 11G shows reduction of angiogenesis in an oxygen-induced retinopathy mouse treated with 10 pg of HSA(C34S)-ARLDDL.

The results of the experiment show that HSA(C34S)-ARLDDL inhibits angiogenesis in an oxygen-induced retinopathy mouse.

### Example 6

### Inhibition of Tumor Growth by HSA(C34S)-ARLDDL

The human PC-3 (prostate cancer) cells were implanted in Non-Obese Diabetic Severe Combined Immune Deficiency (NOD-SCID) mice as follows. Each mouse was injected subcutaneously in the right flank with 1×10⁷ cells. Tumors were monitored every two days. On day 27 of the study, the animals were divided into two groups. One group was treated with saline and another group was treated with HSA(C34S)-ARLDDL (20 mg/kg, intravenously, twice per week).

Tumor size in mm³ was calculated as follows:

Tumor Volume=w²×l/2, where w is width (mm) and l is length (mm) of the tumor.

Tumor weight was estimated based on the assumption that 1 mg is equivalent to 1 mm³ of tumor volume.

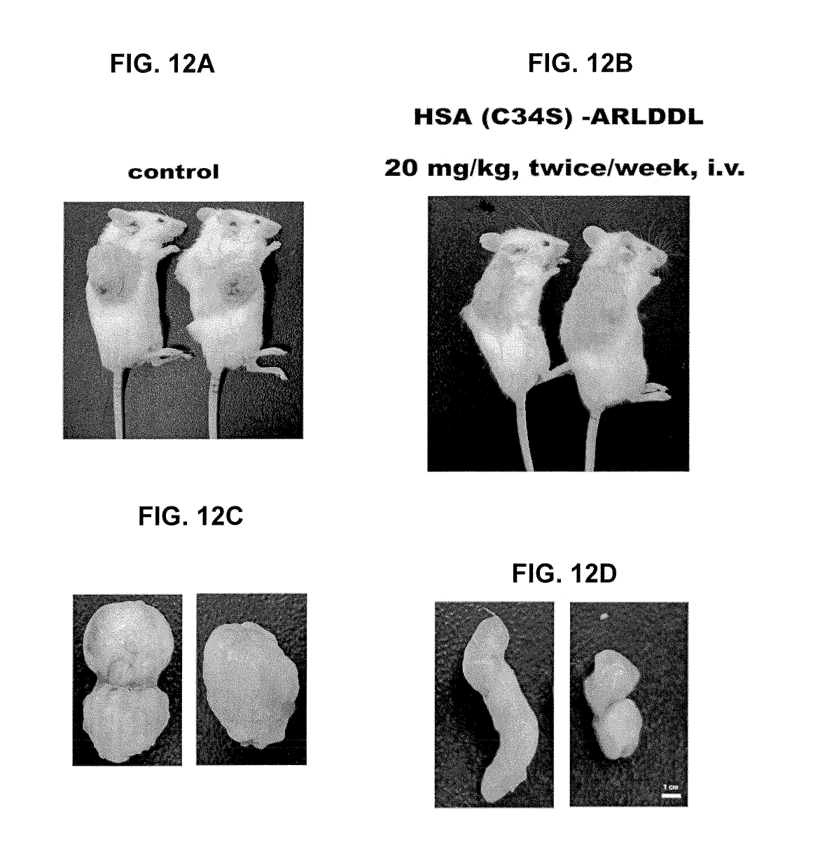

FIG. 12A is a control which shows a photograph of two mice injected with the human PC-3 cells. FIG. 12B is a photograph of two mice injected with the human PC-3 cells and treated with HSA(C34S)-ARLDDL (20 mg/kg, intravenously, twice per week).

FIG. 12C is a photograph of tumors excised from the control mice and FIG. 12D is a photograph of tumors excised from the mice treated with HSA(C34S)-ARLDDL.

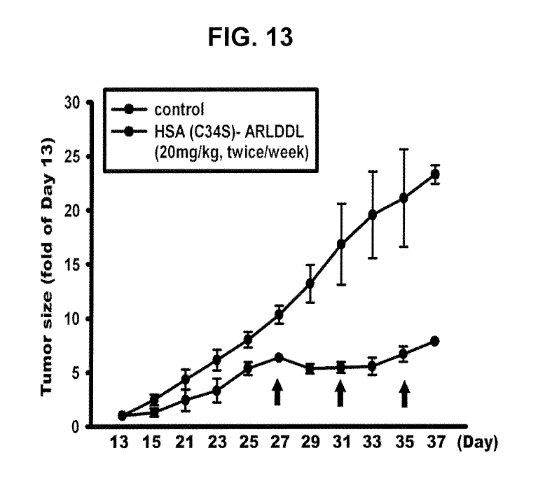

FIG. 13 is graph that shows that HSA(C34S)-ARLDDL significantly inhibited growth of tumors in the mice treated with this protein as measured by tumor size. The arrows on the graph indicate injections of HSA(C34S)-ARLDDL.

### Example 7

### MATRIGEL™ Plug Anti-Angiogenesis Assays

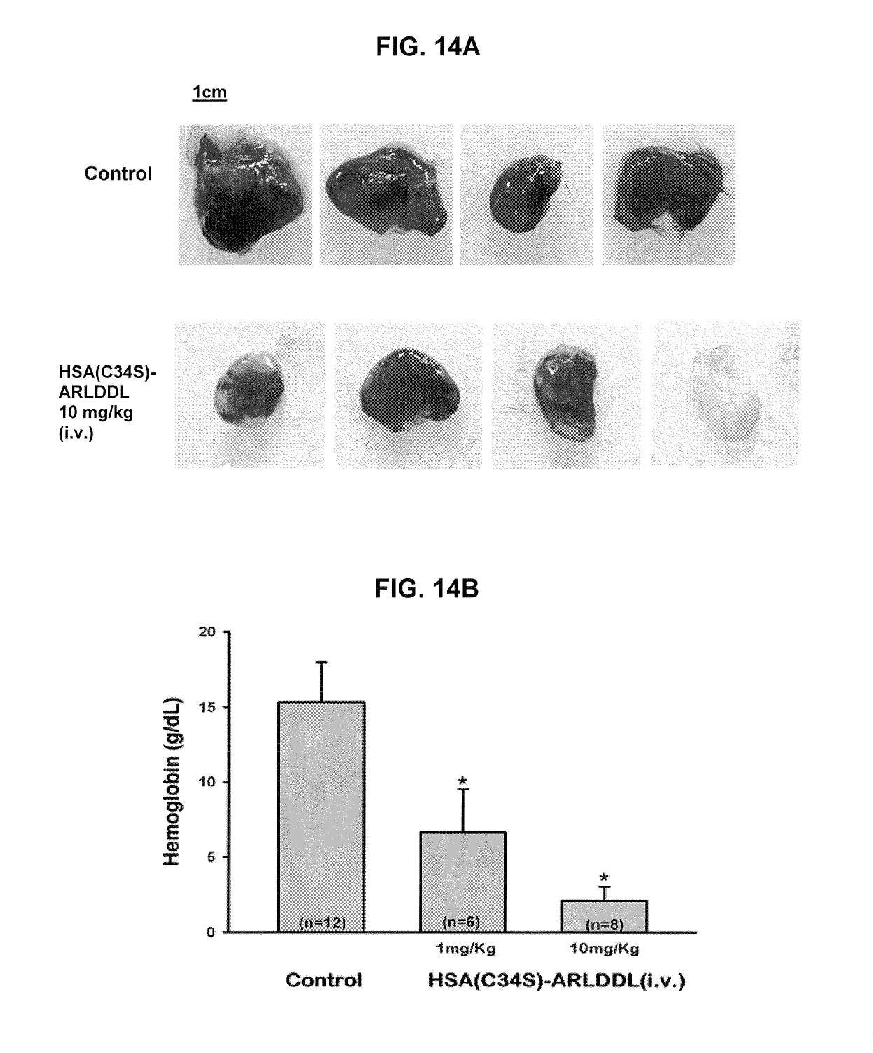

To investigate whether HSA(C34S)-ARLDDL can inhibit angiogenesis, MATRIGEL™ plug angiogenesis assays were used as described in US Patent Application Publication No. 2008-0188413 A1. Briefly, an aliquot (500 μl) of MATRIGEL™ (Becton Dickinson Lab.) containing 200 ng/ml VEGF was injected subcutaneously into the dorsal region of 6-8 week-old C57BL/6 mice. The MATRIGEL™ formed a plug rapidly. HSA(C34S)-ARLDDL at 10 mg/kg or at 1 mg/kg was intravenously administered once on Day 2 and the mice were sacrificed on Day 7. FIG. 14A depicts photographs of the plugs.

Neovessels were quantified by measuring the hemoglobin of the plugs as an indication of blood vessel formation with the Drabkin method and Drabkin reagent kit 525 (Sigma) (FIG. 14B).

As FIGS. 14A and 14B show that HSA(C34S)-ARLDDL was effective in inhibiting angiogenesis using MATRIGEL™ plug assays. *: P<0.05 versus control

### Amino Acid and Nucleotide Sequences Used in the Application

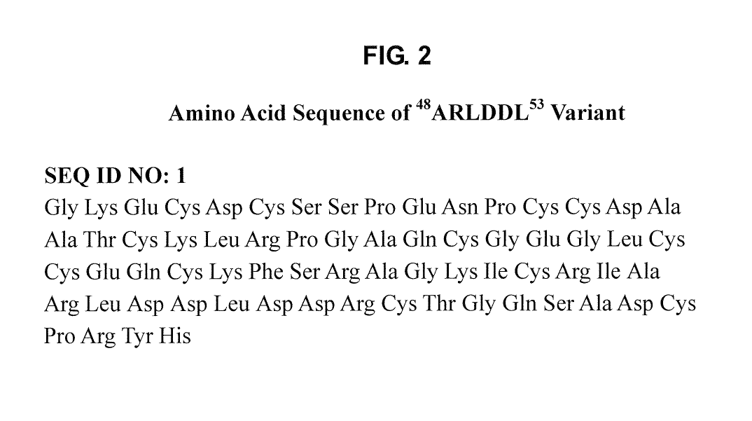

SEQ ID NO: 1 is an amino acid sequence of ARLDDL variant of rhodostomin. It is set forth in FIG. 2.

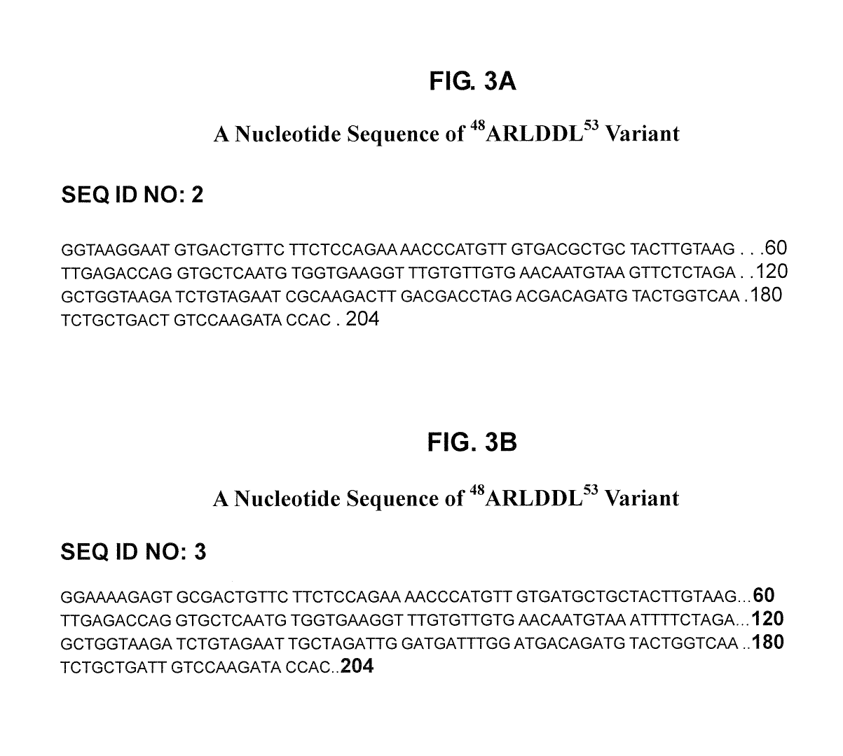

SEQ ID NO: 2 is one nucleotide sequence that encodes ARLDDL variant of rhodostomin. It is set forth in FIG. 3A. SEQ ID NO: 3 is another nucleotide sequence that encodes that encodes ARLDDL variant of rhodostomin. It is set forth in FIG. 3B.

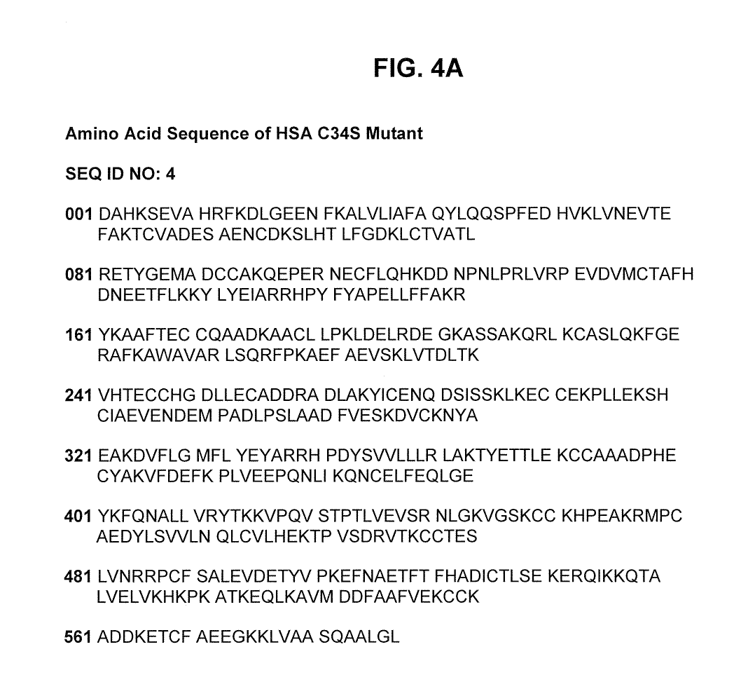

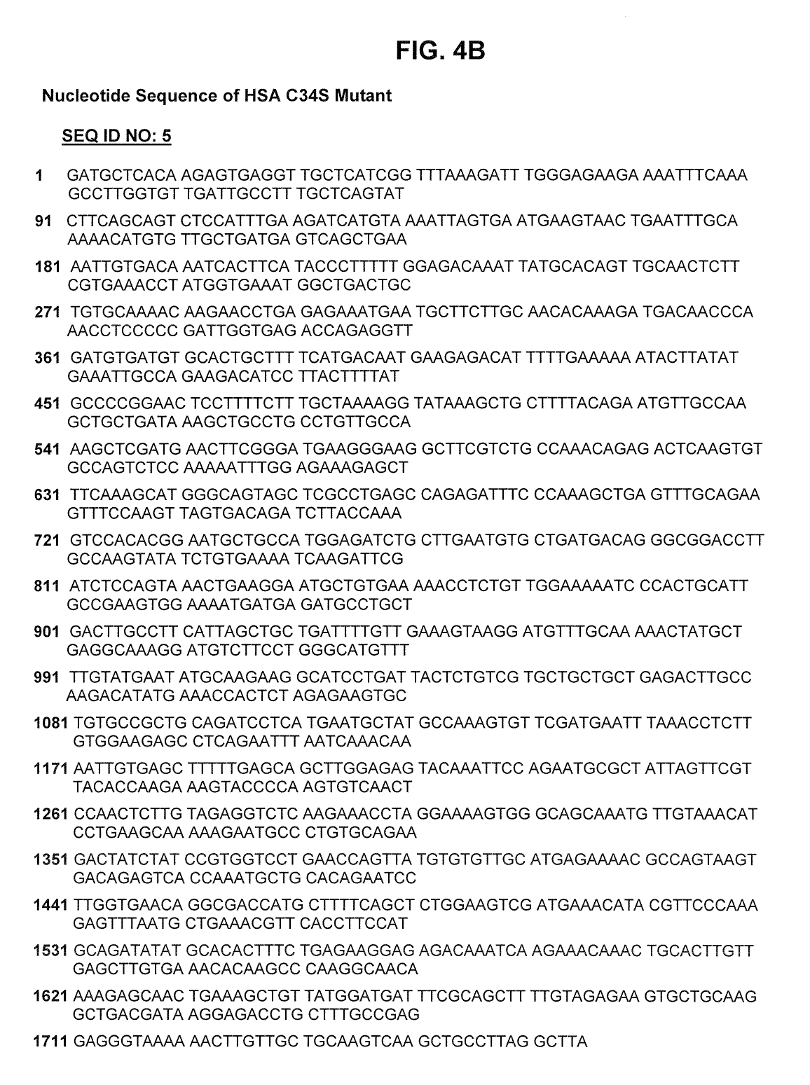

SEQ ID NOS: 4 and 5 are, respectively, amino acid and nucleotide sequences of HSA C34S mutant. They are set forth in FIGS. 4A and 4B.

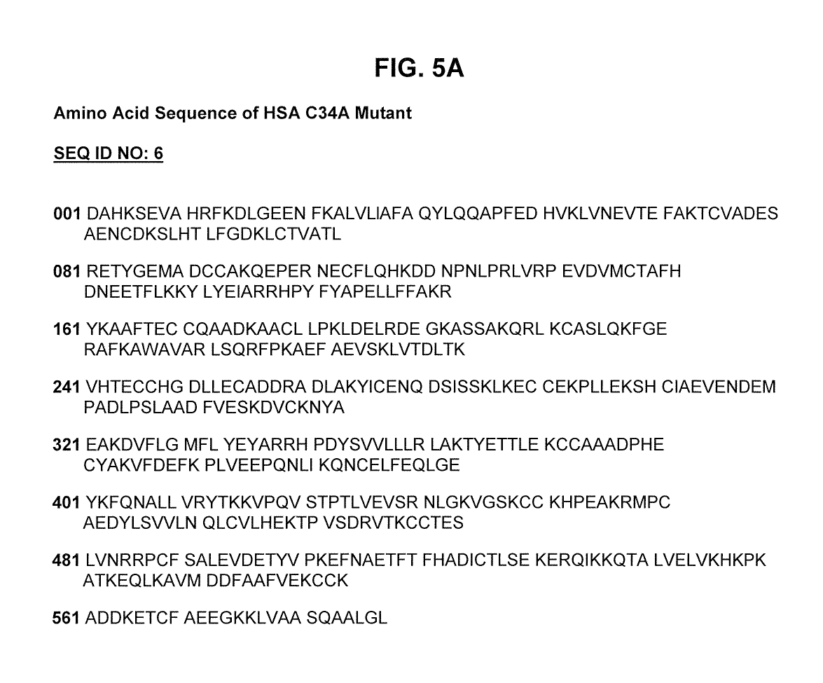

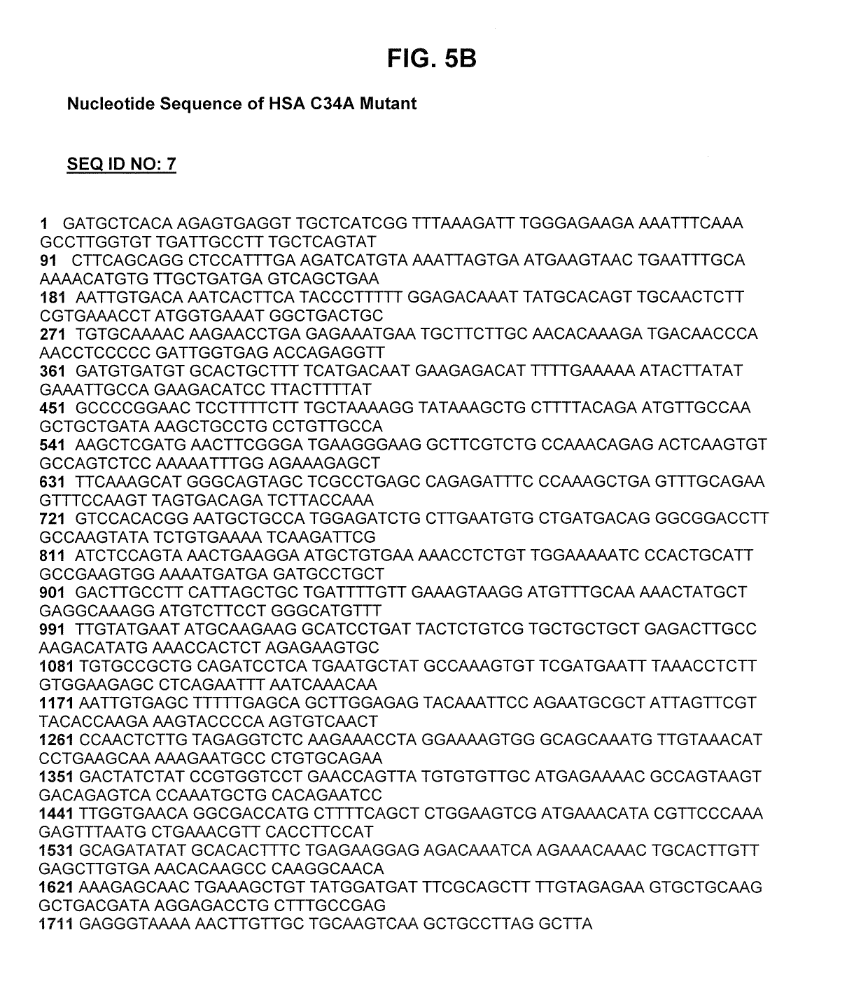

SEQ ID NOS: 6 and 7 are, respectively, amino acid and nucleotide sequences of HSA C34A mutant. They are set forth in FIGS. 5A and 5B.

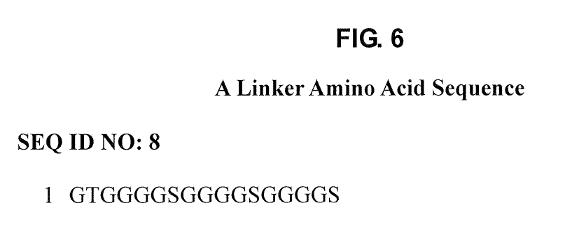

SEQ ID NO: 8 is an amino acid sequence of a linker amino acid. It is set forth in FIG. 6.

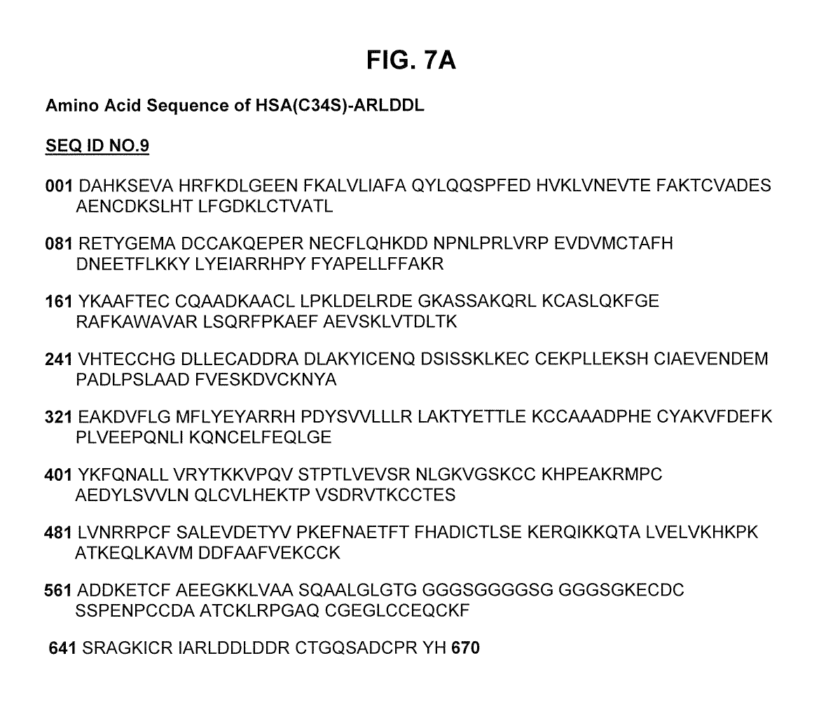

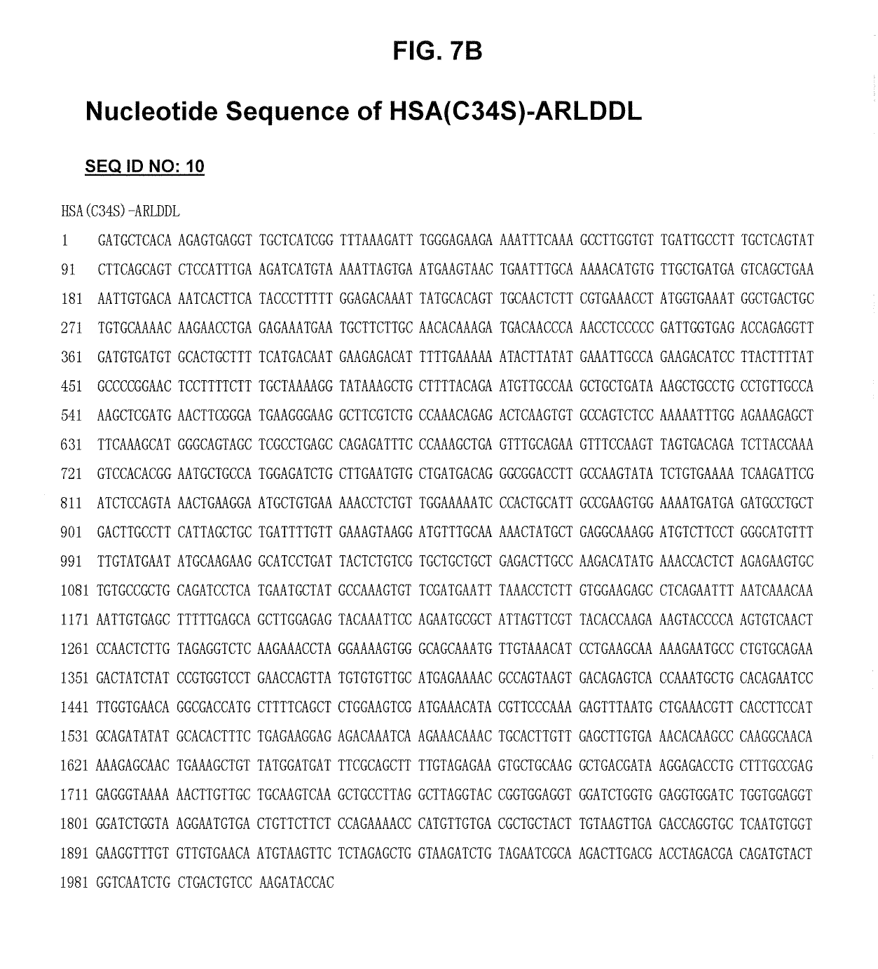

SEQ ID NOS: 9 and 10 are, respectively, amino acid and nucleotide sequences of HSA(C34S)-ARLDDL mutant. They are set forth in FIGS. 7A and 7B.

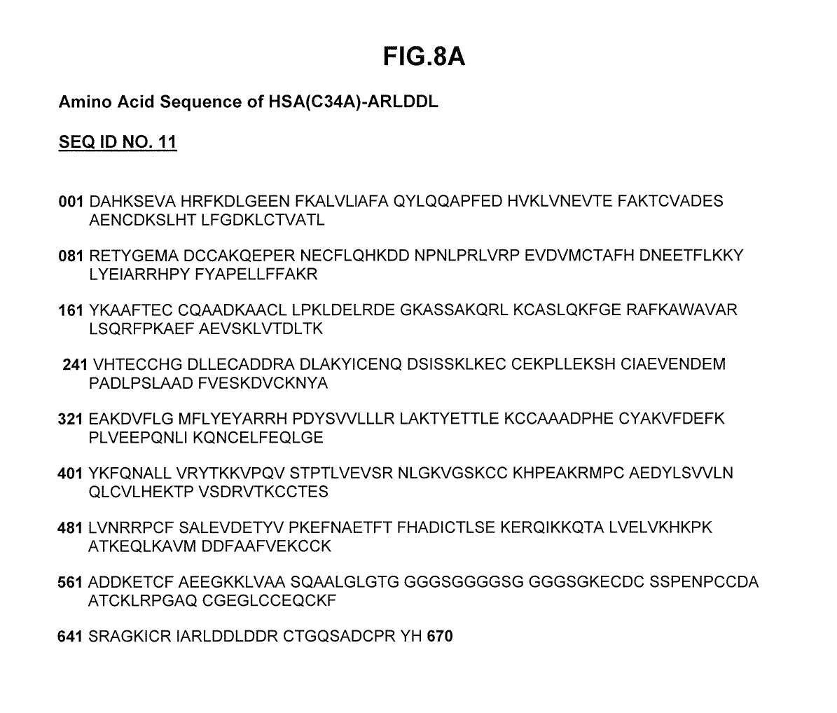

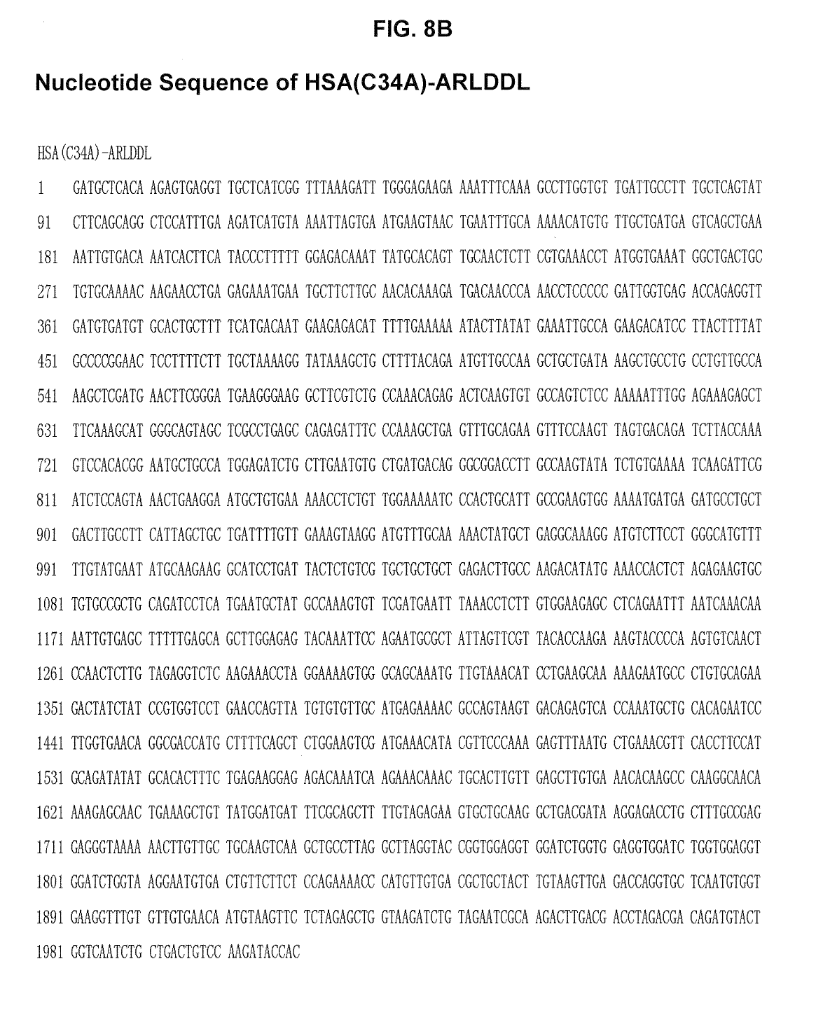

SEQ ID NOS: 11 and 12 are, respectively, amino acid and nucleotide sequences of HSA(C34A)-ARLDDL mutant. They are set forth in FIGS. 8A and 8B.

The foregoing description of the exemplary embodiments of the invention has been presented only for the purposes of illustration and description and is not intended to be exhaustive or to limit the invention to the precise forms disclosed. Many modifications and variations are possible in light of the above teaching.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A polypeptide comprising an amino acid sequence of SEQ ID NO: 1, wherein the polypeptide is conjugated with a variant of human serum albumin (HSA) comprising an amino acid sequence of SEQ ID NO: 4, or a pharmaceutically acceptable salt of said polypeptide.

2. A polypeptide comprising an amino acid sequence of SEQ ID NO: 1, wherein the polypeptide is conjugated with a variant of human serum albumin (HSA) comprising an amino acid sequence of SEQ ID NO: 6, or a pharmaceutically acceptable salt of said polypeptide.

3. The polypeptide of claims 1 or 2, wherein said polypeptide comprises a linker amino acid sequence.

4. The polypeptide of claim 3, wherein said linker amino acid sequence comprises a combination of glycine and serine amino acids.

5. The polypeptide of claim 3, wherein said linker amino acid sequence comprises an amino acid sequence of SEQ ID NO: 8.

6. A polypeptide comprising an amino acid sequence of SEQ ID NO: 9, or a pharmaceutically acceptable salt of said polypeptide.

7. A polypeptide comprising an amino acid sequence of SEQ ID NO: 11, or a pharmaceutically acceptable salt of said polypeptide.

8. A polypeptide encoded by a polynucleotide comprising a nucleotide sequence of SEQ ID NO: 10.

9. A polypeptide is encoded by a polynucleotide comprising a nucleotide sequence of SEQ ID NO: 12.

10. A physiologically acceptable composition comprising the polypeptide of claims 1 or 2, or a pharmaceutically acceptable salt thereof, and a pharmaceutically acceptable carrier.

11. A physiologically acceptable composition comprising a polypeptide comprising an amino acid sequence of SEQ ID NO: 9, or a pharmaceutically acceptable salt of said polypeptide, and a pharmaceutically acceptable carrier.

12. A physiologically acceptable composition comprising a polypeptide encoded by a polynucleotide comprising a nucleotide sequence of SEQ ID NO: 10, or a pharmaceutically acceptable salt of said polypeptide, and a pharmaceutically acceptable carrier.

13. A method for the treatment and/or prevention of an αvβ3 integrin-associated disease comprising administering to a mammal in need thereof a therapeutically effective amount of the polypeptide of claims 1 or 2, or a pharmaceutically acceptable salt thereof.

14. A method for the treatment and/or prevention of an αvβ3 integrin-associated disease comprising administering to a mammal in need thereof a therapeutically effective amount of a polypeptide comprising an amino acid sequence of SEQ ID NO: 9, or a pharmaceutically acceptable salt thereof.

15. A method for the treatment and/or prevention of an αvβ3 integrin-associated disease comprising administering to a mammal in need thereof a therapeutically effective amount of a polypeptide encoded by a polynucleotide comprising a nucleotide sequence of SEQ ID NO: 10, or a pharmaceutically acceptable salt thereof.

16. The method of claims 14 or 15, wherein said αvβ3 integrin-associated disease is tumor growth or tumor metastasis in bone.

17. The method of claims 14 or 15, wherein said αvβ3 integrin-associated disease is an angiogenesis-related eye disease selected from the group consisting of age-related macular degeneration, diabetic retinopathy, corneal neovascularizing diseases, age-related ischaemia-induced neovascularizing retinopathy, high myopia, and retinopathy of prematurity.

18. The method of claims 14 or 15, wherein said αvβ3 integrin-associated disease is selected from the group consisting of osteoporosis, malignancy-induced hypercalcemia, multiple myeloma, and Paget's disease.

19. The method of claims 14 or 15, further comprising co-administering to said mammal another active agent.

20. The method of claim 19, wherein said another active agent is selected from the group consisting of VEGF antagonists, anti-inflammation agents, bisphosphonates, and cytotoxic agents.

21. A method for making the polypeptide according to claims 1 or 2 comprising (a) constructing a gene encoding the polypeptide according to claims 1 or 2, (b) transfecting a host cell with the gene of step (a); (c) growing said host cell in a culture medium; and (d) isolating said polypeptide.

22. A method for making a polypeptide comprising an amino acid sequence of SEQ ID NO: 9 comprising (a) constructing a gene encoding said polypeptide, (b) transfecting a host cell with the gene of step (a); (c) growing said host cell in a culture medium; and (d) isolating said polypeptide.


SEQUENCE LISTING (12 sequences)

SEQ ID NO: 1 - protein, 68 aa, Artificial Sequence
  Description of Artificial Sequence Synthetic rhodostomin variant
GKECDCSSPENPCCDAATCKLRPGAQCGEGLCCEQCKFSRAGKICRIARLDDLDDRCTGQSADCPRYH

SEQ ID NO: 2 - DNA, 204 bp, Artificial Sequence
  Description of Artificial Sequence Synthetic rhodostomin variant
ggtaaggaatgtgactgttcttctccagaaaacccatgttgtgacgctgctacttgtaagttgagaccaggtgctcaatgtggtgaaggtttgtgttgtgaacaatgtaagttctctagagctggtaagatctgtagaatcgcaagacttgacgacctagacgacagatgtactggtcaatctgctgactgtccaagataccac

SEQ ID NO: 3 - DNA, 204 bp, Artificial Sequence
  Description of Artificial Sequence Synthetic rhodostomin variant
ggaaaagagtgcgactgttcttctccagaaaacccatgttgtgatgctgctacttgtaagttgagaccaggtgctcaatgtggtgaaggtttgtgttgtgaacaatgtaaattttctagagctggtaagatctgtagaattgctagattggatgatttggatgacagatgtactggtcaatctgctgattgtccaagataccac

SEQ ID NO: 4 - protein, 585 aa, Artificial Sequence
  Description of Artificial Sequence Synthetic Human Serum Albumin Mutant C34S
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - DNA, 1755 bp, Artificial Sequence
  Description of Artificial Sequence Nucleotide Sequence of HSA C34S Mutant
gatgctcacaagagtgaggttgctcatcggtttaaagatttgggagaagaaaatttcaaagccttggtgttgattgcctttgctcagtatcttcagcagtctccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacgttcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 6 - protein, 585 aa, Artificial Sequence
  Description of Artificial Sequence Synthetic Human Serum Albumin Mutant C34A
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 7 - DNA, 1755 bp, Artificial Sequence
  Description of Artificial Sequence Nucleotide Sequence of HSA C34A Mutant
gatgctcacaagagtgaggttgctcatcggtttaaagatttgggagaagaaaatttcaaagccttggtgttgattgcctttgctcagtatcttcagcaggctccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacgttcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 8 - DNA, 17 bp, Artificial Sequence
  Description of Artificial Sequence Synthetic Linker
gtggggsggggsggggs

SEQ ID NO: 9 - protein, 670 aa, Artificial Sequence
  Description of Artificial Sequence Synthetic HSA(C34S)-ARLDDL Mutant
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGTGGGGSGGGGSGGGGSGKECDCSSPENPCCDAATCKLRPGAQCGEGLCCEQCKFSRAGKICRIARLDDLDDRCTGQSADCPRYH

SEQ ID NO: 10 - DNA, 2010 bp, Artificial Sequence
  Description of Artificial Sequence Nucleotide Sequence of HSA(C34S)-ARLDDL
gatgctcacaagagtgaggttgctcatcggtttaaagatttgggagaagaaaatttcaaagccttggtgttgattgcctttgctcagtatcttcagcagtctccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacgttcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggtaccggtggaggtggatctggtggaggtggatctggtggaggtggatctggtaaggaatgtgactgttcttctccagaaaacccatgttgtgacgctgctacttgtaagttgagaccaggtgctcaatgtggtgaaggtttgtgttgtgaacaatgtaagttctctagagctggtaagatctgtagaatcgcaagacttgacgacctagacgacagatgtactggtcaatctgctgactgtccaagataccac

SEQ ID NO: 11 - protein, 670 aa, Artificial Sequence
  Description of Artificial Sequence Synthetic HSA(C34A)-ARLDDL Mutant
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGTGGGGSGGGGSGGGGSGKECDCSSPENPCCDAATCKLRPGAQCGEGLCCEQCKFSRAGKICRIARLDDLDDRCTGQSADCPRYH

SEQ ID NO: 12 - DNA, 2010 bp, Artificial Sequence
  Description of Artificial Sequence Nucleotide Sequence of HSA(C34A)-ARLDDL Mutant
gatgctcacaagagtgaggttgctcatcggtttaaagatttgggagaagaaaatttcaaagccttggtgttgattgcctttgctcagtatcttcagcaggctccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacgttcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggtaccggtggaggtggatctggtggaggtggatctggtggaggtggatctggtaaggaatgtgactgttcttctccagaaaacccatgttgtgacgctgctacttgtaagttgagaccaggtgctcaatgtggtgaaggtttgtgttgtgaacaatgtaagttctctagagctggtaagatctgtagaatcgcaagacttgacgacctagacgacagatgtactggtcaatctgctgactgtccaagataccac